SEQUENCE LISTING (1 sequence)

SEQ ID NO: 1 - protein, 8 aa, artificial sequence
  peptide that mimics the crosslinking site in alpha 2-plasmin inhibitor (abbreviated TG)
NQEQVSPL

(19) United States
(12) Patent Application Publication
(10) Pub. No.: US 2005/0180957 A1
(43) Pub. Date: Aug. 18, 2005

(54) Method of using fibrin-bound angiogenic factors to stimulate vascularization of transplant site of encapsulated cells

(76) Inventors: David William Scharp, Mission Viejo, CA (US); Paul Presley Latta, Irvine, CA (US); Xiaojie Yu, Irvine, CA (US); Jeffrey Hubbell, Morges (CH)

Correspondence Address: NOVOCELL, INC., 31 TECHNOLOGY DRIVE, SUITE 100, IRVINE, CA, 92618, US

(21) Appl. No.: 11/037,727

(22) Filed: Jan. 18, 2005

Related U.S. Application Data
(60) Provisional application No. 60/537,373, filed on Jan. 16, 2004.

(51) Int. Cl. A61K 45/00; A61K 38/18; A61K 33/06

(52) U.S. Cl. 424/93.7; 514/2; 424/682

(57) ABSTRACT

The present invention relates to compositions and methods of treating a disease, such as diabetes, by implanting encapsulated biological material with a growth factor and conjugate into a patient in need of treatment. Several methods are presented to accomplish transplanting several different types of biological materials. This invention also provides methods of utilizing these encapsulated biological materials to treat different human and animal diseases or disorders by implanting them into several areas in the body including the subcutaneous site.

### RELATED APPLICATIONS

This application claims priority to U.S. Application No. 60/537,373; filed Jan. 16, 2003, which is incorporated herein by reference.

All publications, patents, patent applications, databases and other references cited in this application, all related applications referenced herein, and all references cited therein, are incorporated by reference in their entirety as if restated here in full and as if each individual publication, patent, patent application, database or other reference were specifically and individually indicated to be incorporated by reference.

### BACKGROUND OF THE INVENTION

1. Field of the Invention

The present invention relates generally to cell-based therapy, in particular, methods of implanting cells with sufficient access to oxygen and other nutrients at the implant site by stimulating neovascularization.

2. Description of the Related Art

Pancreatic islet transplantation is an attractive procedure for the treatment of Type I diabetes, with the promise of normal glucose control without the burden of external insulin treatment. This technique still has to overcome problems related to the inconsistencies in the achievement of insulin independence. The islet supply and side effects related to systemic immunosuppression currently restrict the clinical application to a limited number of Type I diabetic patients. The use of a combination product where pancreatic islets are combined with biomaterials in order to provide protection to the graft is an appealing model to overcome some of these problems.

Transplantation of human islets with immunosuppression is done by injecting islets isolated from a cadaver into the portal vein by direct injection percutaneously between the ribs, into the liver, and then the portal vein by fluoroscopic direction. Essentially all of the human islet transplants have been done by this technique, except for the first ones done by umbilical vein injection via a cutdown. A major risk of this procedure is increased portal venous pressures, depending on the rate of infusion and the amount infused, due to the injection of the islets into the portal vein. Also, portal venous thrombosis or hemorrhage is a complication of this procedure.

In addition to injecting the islets into the portal vein, a few patients have had islets injected into the body of the spleen. The spleen is more fragile than the liver so these injections are performed at the time of kidney transplantation so the splenic injection can be done as an open procedure. Freely injecting the islets into the peritoneal cavity has been performed in mouse transplants without difficulty. However, in larger animals or humans, a peritoneal cavity implantation site requires double the quantity of islets than injection into the portal vein. If any rejection or inflammatory reactions occur, then adhesions tend to form between the loops of intestine, as well as, to the omentum. This reaction can lead to additional long-term problems, such as, bowel obstruction.

There are several approaches to encapsulating cells to treat a number of diseases and disorders, without the need for immunosuppression. There are three main types of encapsulation devices, which can best be categorized by describing the form of encapsulation. The three categories are a] macrodevices, b] microcapsules, and c] conformal coatings.

Macrodevices are large devices containing membranes of permselective sheets or tubes, and supporting structures. They contain one or several compartments for the encapsulated cells. They are designed for implantation into extravascular or vascular sites. Some are designed to grow into the host to increase oxygen diffusion across the membranes of these large devices. Others are designed to have no reaction by the host, thus increasing the ease of removal from different sites. There have been two major types of macrodevices developed: a] flat sheet and b] hollow fiber.

Among the flat sheet devices, one type (Baxter, Theracyte) is made of several layers for strength. It has diffusion membranes between support structures with loading ports for replacing the cells. The other type is simpler in design with the device using alginate based membranes and other supporting membranes to encapsulate islets within an alginate matrix between the sheets. The complex device is designed to grow into the body to increase oxygen diffusion. Due to its relatively large size, there are few sites in the body able to accommodate a flat sheet macrodevice to treat a disease like diabetes. The cells inside the device are expected to survive for a finite time. Reloading of new cells are required for this device's long-term application. It has proven quite difficult to flush and reload cells, while at the same time maintaining the critical cell compartment distance for oxygen diffusion.

The second flat sheet style of device is designed to be an “all in/all out” device with little interaction with the host. For the diabetes product, it has been quite difficult to place this device into the intraperitoneal cavity of large animals, while maintaining its integrity. This is due to the difficulty of securing it in the abdomen so the intestines do not cause move or wrinkle the device, which can damage or break it.

The other major macrodevice type is the hollow fiber, which is made by extruding thermoplastic materials. The hollow fibers can be made large enough to act as blood conduits. One model is designed to be fastened into the host's large blood vessels with the encapsulated cells behind a permselective membrane within the device. This type has shown efficacy in large animal diabetic trials, but has been plagued with problems in vascular site access. Thrombosis and hemorrhage complications have caused it abandonment as a clinically relevant product.

Another hollow fiber model is much smaller in diameter and designed as an extravascular device. Low packing densities causes the length of this device to approach many meters. This approach also was abandoned for treating diabetes as not being clinically relevant. Additionally, sealing the open ends of the fiber is not trivial and strength is a problem depending upon the extravascular site.

The microcapsule was the first to offer potential clinical efficacy. Encapsulated islets in alginate microcapsules eliminated diabetes in rodents when implanted intraperitoneally. However, nearly 25 years have passed without a demonstration of clinical efficacy. One of the problems associated with microcapsules is their relatively large size in combination with low packing densities of cells, especially for the treatment of diabetes. Another is the use of alginate, an ionically crosslinked hydrogel dependent upon the calcium concentration for its degree of crosslinking. The permselectivity of pure alginate capsules has been difficult to control with most having a wide open molecular weight cutoff. Varieties of positively charged crosslinked agents, such as polylysine, have been used as a second coating on the capsule to provide permselectivity. However, polylysine and most other similar molecules cause an inflammatory reaction. This requires an additional third coating of alginate to reduce the host's response to the capsule. In addition, it has been difficult to produce very pure alginates that are not reactive within the host after implantation. Trying to reduce the size of the alginate microcapsules causes two major problems: first, very large quantities of empty capsules without any cells, and second, poorly coated cells. There is no force to keep the cells within the center of the microcapsule, so the amount of incomplete coatings goes up exponentially with a decrease in the size of the capsules. Production of conformal coatings has not been demonstrated with alginate microcapsules.

The last category of cell encapsulation is conformal coating. A conformally coated cell aggregate is one that has a substantially uniform cell coating around the cell aggregate regardless of its size or shape. This coating not only may be uniform in thickness, but it also may be uniform in the protective permselective nature of the coating that provides uniform immune protection. Furthermore, it may be uniform in strength and stability, thus preventing the coated material from being violated by the host's immune system.

### SUMMARY OF THE INVENTION

This invention combines the implantation of cells, tissues or organs [stem cell, autologous, allogeneic, xenogeneic or genetically-modified], either unencapsulated, or encapsulated in macrodevices, microcapsules, or conformal coatings in an implant site combined with fibrin glue production, or its equivalent, and conjugated angiogenic growth factors to enhance survival and function of the implanted cells, tissues or organs at these sites.

In one embodiment the invention is directed to a method of stimulating vascularization at a transplant site in an animal which includes providing a pharmaceutical composition including a fibrinogen, a modified angiogenic growth factor, one or more encapsulated cells, a thrombin and a divalent salt; and administering the pharmaceutical composition at said transplant site to said animal. In preferred embodiments, the angiogenic growth factor is a modified angiogenic growth factor. More preferably, the divalent salt is a calcium salt. In preferred embodiments, the administration of the thrombin/encapsulated cell/fibrinogen/modified-angiogenic growth factor solution is by injection. Preferably, the animal is from the Class Mammalia. More preferably, the animal is Human.

Preferably, the angiogenic growth factor is Angiogenin, Angiotropin, Epidermal Growth Factor (EGF), Beta Fibroblast Growth Factor (β-FGF), Fibroblast Growth Factor-2 (FGF-2), Fibroblast growth factors (FGFs), Heparin-binding EGF-like growth factor, Hepatocyte growth factor (HGF), Insulin-Like Growth Factor I (IGF-I), Interferon-γ (IFN-γ), Interferon-g-inducible protein-10 (IP-10), Interleukin-8 (IL-8), Macrophage inflammatory protein-1 (MIP-1), Placental growth factor (PIGF), Platelet Derived Endothelial Cell Growth Factor, Platelet factor-4 (PF-4), Platelet-derived growth factor (PDGF), platelet-derived growth factor-BB (PDGF-BB), Pleiotrophin, Transforming Growth Factor α (TGF-α), Transforming Growth Factor β (TGF-β), or Vascular Endothelial Growth Factor (VEGF). More preferably, the angiogenic growth factor is VEGF.

In preferred embodiments, the encapsulated cell is a macroencapsulated cell, a microencapsulated cell or a conformally coated encapsulated cell Preferably, the encapsulated cell is a derived cell from a stem cell. In preferred embodiments, the derived cell is a hormone-producing cell. More preferably, the hormone-producing cell is an insulin-producing cell. Preferably, the encapsulated cell is from the Class Mammalia. More preferably, the encapsulated cell is Human.

Some embodiments may also include the step of administering an immunosuppressant or anti-inflammatory agent, either alone or in combination. Preferably, administration of the immunosuppressant or anti-inflammatory agent, either alone or in combination, is for a period of no more than 6 months from the time of treatment. More preferably, administration of the immunosuppressant or anti-inflammatory agent, either alone or in combination, is for a period of no more than 1 month from the time of treatment.

In preferred embodiments, the pharmaceutical composition additionally comprises Factor XIII. In a most preferred embodiment, a substrate for a transglutaminase activity of Factor XIII is attached to the angiogenic growth factor.

In one embodiment, the invention is directed to a method of preparing a pharmaceutical composition including the steps of:
(a) preparing a solution comprising a fibrinogen, a fibrinolysis inhibitor, and an angiogenic growth factor to form a fibrinogen/fibrinolysis inhibitor/angiogenic growth factor solution;
(b) adding at least one encapsulated cell to the fibrinogen/fibrinolysis inhibitor/angiogenic growth factor solution; and
(c) adding a thrombin into the encapsulated cell/fibrinogen/fibrinolysis inhibitor/angiogenic growth factor solution of step (b) to produce a pharmaceutical composition which includes a thrombin, at least one encapsulated cell, fibrinogen, fibrinolysis inhibitor, and angiogenic growth factor.

In a preferred embodiment, the invention is directed to a method of stimulating vascularization at a transplant site in an animal which includes administering the thrombin/encapsulated cell/fibrinogen/modified-growth factor solution prepared by the method as described above to an animal. In preferred embodiments, the solution of step (a) additionally includes Factor XIII. Preferably, the thrombin added in step (c) is in a solution which includes additionally a divalent salt. More preferably, the divalent salt is calcium.

Some embodiments are directed to preparing a pharmaceutical composition which includes the steps of:
(a) preparing a solution which includes a thrombin and an angiogenic growth factor to form a thrombin/angiogenic growth factor solution;
(b) adding at least one encapsulated cell to the thrombin/angiogenic growth factor solution; and
(c) adding a fibrinogen and a fibrinolysis inhibitor into the encapsulated cell/thrombin/angiogenic growth factor solution of step (b) to produce a pharmaceutical composition which includes a fibrinogen, a fibrinolysis inhibitor, at least one encapsulated cell, thrombin and an angiogenic growth factor.

Some embodiments are directed to a method of stimulating vascularization at a transplant site in an animal which includes administering the pharmaceutical composition prepared as described above to said animal. Preferably, the thrombin added in step (a) is in a solution which also includes a divalent salt. More preferably, the divalent salt is a calcium salt. In some preferred embodiments, the solution of step (c) also includes Factor XIII.

Some embodiments of the invention are directed to a method of stimulating vascularization at a transplant site in an animal which includes placing a first solution which includes thrombin, angiogenic growth factor and at least one encapsulated cell into a first barrel of a syringe; placing a second solution which includes fibrinogen and fibrinolysis inhibitor into a second barrel of a syringe; and injecting the first and second solutions into an injection site on said animal.

In preferred embodiments, the injection site on the animal is subcutaneous, intraperitoneal, intramuscular, intra-omental, or into an organ. More preferably, the injection site on the animal is a subcutaneous site.

Embodiments of the invention are directed to a pharmaceutical composition which includes a fibrinogen, an angiogenic growth factor, one or more encapsulated cells, a thrombin and a divalent salt. Preferably, the angiogenic growth factor is Angiogenin, Angiotropin, Epidermal Growth Factor (EGF), Beta Fibroblast Growth Factor (β-FGF), Fibroblast Growth Factor-2 (FGF-2), Fibroblast growth factors (FGFs), Heparin-binding EGF-like growth factor, Hepatocyte growth factor (HGF), Insulin-Like Growth Factor I (IGF-I), Interferon-γ (IFN-γ), Interferon-g-inducible protein-10 (IP-10), Interleukin-8 (IL-8), Macrophage inflammatory protein-1 (MIP-1), Placental growth factor (PIGF), Platelet Derived Endothelial Cell Growth Factor, Platelet factor-4 (PF-4), Platelet-derived growth factor (PDGF), platelet-derived growth factor-BB (PDGF-BB), Pleiotrophin, Transforming Growth Factor α (TGF-α), Transforming Growth Factor β (TGF-β), or Vascular Endothelial Growth Factor (VEGF). In some preferred embodiments, the angiogenic growth factor is a modified angiogenic growth factor. More preferably, the modified angiogenic growth factor is α-2 PI₁₋₈-VEGF₁₂₁.

Preferably, the encapsulated cell is a macroencapsulated cell, a microencapsulated cell or a conformally coated encapsulated cell. Preferably, the divalent salt is a calcium salt.

In preferred embodiments, the encapsulated cell is a cell derived from a stem cell. More preferably, the derived cell is a hormone-producing cell. Yet more preferably, the hormone-producing cell is an insulin-producing cell.

In preferred embodiments, the encapsulated cell is from the Class Mammalia. More preferably, the encapsulated cell is human.

Further aspects, features and advantages of this invention will become apparent from the detailed description of the preferred embodiments which follow.

### BRIEF DESCRIPTION OF THE DRAWINGS

FIG. 1 is a graphical representation of the Blood Glucose levels (mg/dL) and Insulin requirements in a streptozotocin-induced diabetic baboon with a subcutaneous implant of an encapsulated islet allograft and 30 days of low dose cyclosporine and Metformin [♦=Blood Glucose, ●=Insulin].

FIG. 2 is a graphical representation of Glycated Hemoglobin A1c in a streptozotocin-induced diabetic baboon with a subcutaneous implant of an encapsulated islet allograft and 30 days of low dose cyclosporine and Metformin.

FIG. 3 shows photomicrographs of the encapsulated islet allografts at the subcutaneous implant site in a streptozotocin-induced diabetic baboon with 30 days of low dose cyclosporine and Metformin [histological staining for insulin or VEGF receptor]. The encapsulated islet allografts from the first implantation are shown at 20 months and the encapsulated islet allografts from the second implantation are shown at 5 months.

FIG. 4 is a graphical representation of the Blood Glucose levels (mg/dL) and Insulin requirements in a streptozotocin-induced diabetic baboon with a subcutaneous implant of an encapsulated islet allograft and 30 days of low dose cyclosporine [♦=Blood Glucose, ●=Insulin].

FIG. 5 is a graphical representation of Glycated Hemoglobin A1c in a streptozotocin-induced diabetic baboon with a subcutaneous implant of an encapsulated islet allograft and 30 days of low dose cyclosporine.

FIG. 6 is photomicrographs (20×) of the histology of the subcutaneous implant site in Streptozotocin Induced Diabetic Baboon with an encapsulated islet allograft with Fibrin Glue/TG-VEGF₁₂₁ and 30 days of low dose cyclosporine.

FIG. 7 is photomicrographs (100×) of the histology of the subcutaneous implant site in Streptozotocin Induced Diabetic Baboon with an encapsulated islet allograft with Fibrin Glue/TG-VEGF₁₂₁ and 30 days of low dose cyclosporine.

FIG. 8 is a graphical representation of the Blood Glucose levels (mg/dL) and Insulin requirements in a streptozotocin-induced diabetic baboon with a subcutaneous implant of an encapsulated islet allograft with Fibrin Glue/TG-VEGF₁₂₁ and 30 days of low dose cyclosporine ●=Blood Glucose, ▪=Insulin].

FIG. 9 is a graphical representation of Glycated Hemoglobin A1c in a streptozotocin-induced diabetic baboon with a subcutaneous implant of an encapsulated islet allograft with VEGF-fibrin glue and 30 days of low dose cyclosporine.

FIG. 10 is a graphical representation of the Blood Glucose levels during an Oral Glucose Tolerance Test in a streptozotocin-induced diabetic baboon with a subcutaneous implant of an encapsulated islet allograft with Fibrin Glue/TG-VEGF₁₂₁ and 30 days of low dose cyclosporine.

FIG. 11 is a graphical representation of the C-Peptide Values during an Oral Glucose Tolerance Test in a streptozotocin-induced diabetic baboon with a subcutaneous implant of an encapsulated islet allograft with Fibrin Glue/TG-VEGF₁₂₁ and 30 days of low dose cyclosporine.

FIG. 12 shows photomicrographs of the encapsulated islet allografts at the subcutaneous implant site at 13-14 months in a streptozotocin-induced diabetic baboon with VEGF-fibrin glue and 30 days of low dose cyclosporine [histological staining for insulin]. A—10× of animal in FIG. 8. B—20× of animal in FIG. 7. C—4× of animal in FIG. 12. D—20× of animal in FIG. 13.

FIG. 13 is a graphical representation of the Blood Glucose levels (mg/dL) and Insulin requirements in a streptozotocin-induced diabetic baboon with a subcutaneous implant of an encapsulated islet allograft with Fibrin Glue/TG-VEGF₁₂₁ and 30 days of low dose cyclosporine ●=Blood Glucose, ▪=Insulin].

FIG. 14 is a graphical representation of Glycated Hemoglobin A1c in a streptozotocin-induced diabetic baboon with a subcutaneous implant of an encapsulated islet allograft with VEGF-fibrin glue and 30 days of low dose cyclosporine.

FIG. 15 is a graphical representation of the Blood Glucose levels during an Oral Glucose Tolerance Test in a streptozotocin-induced diabetic baboon with a subcutaneous implant of an encapsulated islet allograft with VEGF-fibrin glue and 30 days of low dose cyclosporine.

FIG. 16 is a graphical representation of the C-Peptide Values during an Oral Glucose Tolerance Test in a streptozotocin-induced diabetic baboon with a subcutaneous implant of an encapsulated islet allograft with VEGF-fibrin glue and 30 days of low dose cyclosporine.

### DETAILED DESCRIPTION OF THE PREFERRED EMBODIMENT

While the described embodiment represents the preferred embodiment of the present invention, it is to be understood that modifications will occur to those skilled in the art without departing from the spirit of the invention. The scope of the invention is therefore to be determined solely by the appended claims.

An important aspect to the feasibility of using encapsulation methods is the relevant size and implant site needed to obtain a physiological result of about 15,000 IEQ/kg-BW. Assuming that injection of isolated islets into the Portal Vein requires about 2-3 ml of pack cells, then a macrodevice consisting of a flat sheet that is one islet thick (˜500 μm) requires a surface area equivalent to 2 US dollar bills. A macrodevice consisting of hollow fibers with a loading density of 5% would need 30 meters of fiber. Alginate microcapsules with an average diameter of 400-600 μm would need a volume of 50-170 ml. However, PEG conformal coating of islets, which produces a 25-50 μm thick covering, would only need a volume of 15-20 ml. These encapsulated cells could be injected into almost any area in the body.

Individual encapsulation of islets with a coating that is selectively permeable (micro-encapsulation) [eliminating the need for a life-long regimen of immunosuppressive drugs] appears to be the most promising approach for islet transplantation. Coating materials must allow diffusion of nutrients, hormones, oxygen, and ions; allow glucose to reach the islets; and permit the insulin released by the beta cells to be readily passed out of the capsule as well as the metabolic waste. Additionally, the coating material must provide an impermeable barrier to larger molecules and cells of the body's immune system. Immunoglobulins and cytokines are prevented from diffusing through the pores in the membrane of properly coated islets. Although the immunoisolation membrane prevents passage of the immune cells and complement, antigens released by the transplanted cells can penetrate the membrane and activate the immune cells, resulting in the release of lymphokines, such as IL-1, and cytotoxic agents, such as free radicals, nitric oxide, and peroxides. However, encapsulated islets must have a low tissue density in order to survive the limited diffusion flux of oxygen. The lower concentration of encapsulated tissue results in a corresponding lower concentration of shed antigens with a reduced concentration of soluble immune agents.

Encapsulation methods have been successfully developed for the islet tissue to maintain viability in the host, provide complete coverage of the islet, allow good insulin release dynamics, allow adequate oxygen diffusion to all cells in the capsule, and be permeable to small and medium-sized proteins while providing a barrier to the larger antibody molecules.

The implantation site of islets is another important issue, where investigations are underway in finding the “optimal” site where the islets can engraft and efficiently start releasing hormones. It is necessary to weigh issues such as the safety and possibility of re-transplantation (peritoneal cavity, subcutaneous transplantation) against proximity to the circulation (intrahepatic transplantation or membranes supporting vascularization). The portal system has been chosen for human islet transplants with relative success, but is still hampered by complications like hepatic hemorrhage and portal vein thrombosis. The ability to perform encapsulated islet implants into the subcutaneous site would significantly reduce the complications associated with these other sites. Intramuscular and subcutaneous sites are especially practical with regard to easy approach, but the lack of vascularization and islet engraftment has made these sites less appealing than other sites. This problem is especially important since the isolation of the islets leads to a total or partial loss of their original vasculature. The revascularization of the transplanted islets is required to ensure their survival.

Attempts at subcutaneous implantation of encapsulated islets have not produced sustainable results in the treatment of diabetes due to some or all of the scientific challenges described above. Kawakami et al. (Cell Transplantation 1997 6, 5:541-545) implanted pancreatic beta cells, encapsulated in agarose-PSSa, subcutaneously in rats. Insulin secretion from the cells was maintained after transplantation. However, this study only examined subcutaneous implantation of the encapsulated islet cells over a one-week period. No evidence has been provided that the insulin secretion response of the cells could be maintained long term in a subcutaneous implant. A treatment site must permit a long term response from the transplanted cells to be feasible for clinical use. The implanted cells must be viable with a high efficiency for years before a method is considered successful.

Kawakami et al. (Transplantation 2002, 73,122-129) enclosed rat islets in an agarose/poly(styrene sulfonic acid) mixed gel and implanted the encapsulated cells into a prevascularized subcutaneous site. This method of obtaining vascularization at an implant site is not feasible for human clinical use. This procedure would require several operations which would be time consuming and increase the risk of infection or inflammation.

Stockley et al. (J. Lab. Clin. Med. 135:484-492) encapsulated allogenic MDCK cells engineered to secrete human growth hormone in alginate-poly-L-lysine-alginate and implanted them subcutaneously. Stockley provides no information about the actual volume of encapsulated cells that are applied. The encapsulated cells can be estimated to have diameters of approximately 1.5 mm (as compared to conformal coated encapsulated cells with diameters of 200 to 250 μm). It is assumed that the capsule volume is 100 μl and does not comprise components other than the encapsulated cells. However one skilled in the art would be unable to determine the desired volume of encapsulated cells needed to administer to a subject.

The present invention relates to methods and compositions that provide sufficient revascularization such that use of subcutaneous sites for implantation of encapsulated islets is a practical option. Another factor of the encapsulation method is the relative diffusion distance between the encapsulated cells and the host. A critical diffusive agent for cell survival is oxygen. These diffusion distances should be minimal since the starting partial pressure of oxygen is 30-40 mm Hg at the tissue level in the body. There is little tolerance for a reduction in diffusive distances, due to the initially low oxygen partial pressure. This would further lower the oxygen concentration to a point where the cells cannot adequately function or survive.

Oxygen supply limitations can have deleterious effects on viability and functionality of encapsulated islets. This can be a contributing or primary cause for poor performance, or for failure of an implant. Theoretical predictions based on oxygen diffusion and consumption models agree with experimental data for (1) size of nonviable core in single islet culture, and (2) loss of viability in high-density culture. As islet loading density increases in a planar diffusion chamber, viable volume fraction decreases, and fully functional volume fraction drops dramatically.

Abundant vascularization at the transplantation site is essential for nutrient exchange and is a prerequisite for cell survival. Dr. Vivek Dixit at UCLA showed that subcutaneously transplanted isolated hepatocytes did not survive for more than a few hours post-transplantation because subcutaneous sites in the body are not characteristically “highly vascularized”.

Islets in the pancreas are normally richly vascularized, but when isolated are completely dependent upon oxygen diffusion from the surrounding media or buffer. Moreover, when encapsulated and transplanted, they are dependent upon oxygen delivered from adjacent capillaries in the transplant site. It is essential to consider both the loss of cells from hypoxia and reduced insulin production from surviving cells receiving only a marginal oxygen supply. One must consider not just single islets but also the competition for oxygen by groups of islets to appreciate the complexity of oxygen needs in tissue culture. For example, a single islet of 200 μm in diameter in tissue culture will have some necrosis of the central core of beta cells. An islet, the same size or even smaller, surrounded by other islets would be even more oxygen-starved. The health of cultured islets should be evaluated because it seems likely (although not yet proven) that islets with hypoxia stress will do worse after encapsulation and transplantation.

Local, controlled induction of angiogenesis remains a challenge that limits tissue-engineering approaches to replace or restore diseased tissues. Development of new therapeutic approaches that stimulate the body's natural mechanisms of new blood vessel formation (angiogenesis) is one of the most active areas of tissue engineering. Targeted delivery of potent inducers of physiological angiogenesis, e.g. vascular endothelial growth factor (VEGF), from polymeric vehicles presents an inviting strategy to improve regional blood flow. New knowledge on side effects adverse to healing caused by VEGF overdose indicate that these approaches may require development of new schemes that permit tight and highly localized regulation of VEGF exposure to induce vasculature with normal morphology and function. Presented is a methodology of therapeutic implants with characteristics that permit sustained liberation of VEGF precisely at the healing site under the local control of the matrix-remodeling proteases (fibrinolysis enzyme(s)) present at the cell surface. First, engineered hydrogels based on the natural biopolymer fibrin that is clinically applied as “fibrin glue”. Second, completely synthetic copolymers made of bioactive peptides and polyethylene glycol (PEG) that are designed as mimetics of natural wound repair matrices such as fibrin or collagen. Covalent conjugation of mutant VEGF proteins to these biomatrices provides retention of the factor until VEGF becomes released by cellular proteolytic activity and matrix degradation.

In some embodiments, a type of modified angiogenic growth factor is used in which a substrate for the transglutaminase activity of Factor XIIIa is attached to the growth factor. The terms “Factor XIII” and “Factor XIIIa” are used interchangeably herein. Factor XIII is the inactive precursor for Factor XIIIa. Any sequence that provides a substrate for Factor XIIIa can be used in these embodiments. In a preferred such embodiment, this sequence comprises a specific sequence from α-2-plasmin inhibitor (α-2PI₁₋₈) that provides a glutamine substrate. This exact sequence has been identified as NQEQVSPL (SEQ ID NO: 1), with the first glutamine being an active amino acid for crosslinking. This permits the growth factor to be crosslinked to a matrix that comprises fibrin or another substrate for Factor XIIIa.

A number of other proteins have also been shown to serve as a substrate for the transglutaminase activity of Factor XIIIa. The glutamine substrate from these proteins can also be attached to the growth factor. Factor XIIIa has been shown to crosslink fibronectin to fibronectin (Barry & Mosher, J. of Biol. Chem., 264:4179-4185, 1989), as well as fibronectin to fibrin itself (Okada, et al., J. of Biol. Chem., 260:1811-1820, 1985). This enzyme also crosslinks von Willebrand factor (Hada, et al., Blood, 68:95-101, 1986). Any site that serves as a substrate for Factor XIIIa can be used according to the invention. In one particular example, a sequence from α-2PI₁₋₈ is attached to VEGF to provide a modified growth factor. In a preferred embodiment, this modified growth factor is prepared as a fusion protein by recombinant methods which are well known in the art. The modified growth factor may be incorporated into the matrix by the action of Factor XIIIa. In preferred embodiments, Factor XIIIa-mediated covalent conjugation of α-2PI₁₋₈-VEGF₁₂₁ to fibrin implants protects VEGF from unregulated burst release and clearance. This technology is described in U.S. patent applications Ser. Nos. 10/024918 and 10/650509, hereby incorporated by reference in their entirety.

The characteristics of these two types of implant matrices permit exploitation of natural proteolytic programs of tissue repair to controllably liberate a potent angiogenic stimulus from the implant matrix while simultaneously forming new vascularized tissue in place of the material.

Cells interact with their environment through protein-protein, protein-oligosaccharide and protein-polysaccharide interactions at the cell surface. Extracellular matrix proteins provide a host of bioactive signals to the cell. This dense network is required to support the cells, and many proteins in the matrix have been shown to control cell adhesion, spreading, migration and differentiation. Some of the specific proteins that have been shown to be particularly active include laminin, vitronectin, fibronectin, fibrin, fibrinogen and collagen.

Some of the specific sequences have been identified that directly interact with cellular receptors and cause either adhesion, spreading or signal transduction so only short active peptide sequences have to be used in in vivo and in vitro experiments.

Work has been done in crosslinking bioactive peptides to large carrier molecules and incorporating them within fibrin gels. The rate of diffusion out of the fibrin gel will be slowed down by attaching the peptides to the large carrier polymers.

Very little work has been done regarding incorporating peptide sequences and other bioactive factors into fibrin gels, and even less has been done regarding covalently binding peptides directly to fibrin. However, a significant amount of energy has been spent on determining which proteins bind to fibrin via enzymatic activity and often in determining the exact sequence, which binds, as well. The sequence for fibrin y-chain crosslinking has been determined, and the exact site has been located. Factor XIIIa has also been shown to crosslink fibronectin to fibronectin, as well as, fibronectin to fibrin itself. Thus, many substrates for Factor XIIIa exist, and a number of these have been identified in detail.

Bidomain proteins and peptides, either formed synthetically or recombinantly, contained both a transglutaminase substrate domain, such as a Factor XIIIa substrate domain, and a bioactive factor. These proteins and peptides are covalently attached to a matrix, such as fibrin, which has a three-dimensional structure capable of supporting cell growth. In the preferred embodiment, the matrix is fibrin. The bioactive factor is preferably a growth factor, such as VEGF, growth factors from the TGF-β superfamily, PDGF, human growth hormone, IGF, and ephrin. Particularly preferred growth factors are TGF-β1, BMP 2, VEGF₁₂₁ and PDGF AB.

There are numerous applications for these matrices that are derivatized with a bioactive factor. Methods described herein incorporate an active sequence or entire factor into the gels to create gels that possess specific bioactive properties.

In the preferred embodiment, the matrix is formed of proteins, most preferably proteins naturally present in the patient into which the matrix is to be implanted. The most preferred protein is fibrin. Fibrin provides a suitable three-dimensional structure for tissue growth and is a native matrix for tissue healing. Other proteins, such as collagen, and glycoproteins, and polysaccharides may also be used or included with fibrin. In some embodiments, it is also possible to use synthetic polymers, such as PEG, that are crosslinkable by ionic or covalent binding. A recombinant form of fibrinogen can be used to form the fibrin network. In some embodiments, the matrix material may include laminin, vitronectin, fibronectin, and/or fibrinogen. In a preferred embodiment, fibrinogen is included in the matrix material and thrombin is used to activate fibrinogen to fibrin.

The matrix material is crosslinkable, and may form a gel. A gel is a material in which a crosslinked polymer network is swollen to a finite extent by a continuous phase of an aqueous solution. The matrix material is preferably biodegradable by naturally present enzyme.

Many different types of bioactive factors can be linked to the matrix. Vascular endothelial growth factor (VEGF) is a key factor in endothelial cell biology and blood vessel formation and a candidate therapeutic for the stimulation of angiogenesis-dependent tissue regeneration. The objective of this study was to test the angiogenic activity of VEGF₁₂₁ of the biomaterial fibrin, a natural substrate for endothelial cell growth and clinically accepted as “fibrin glue”. To achieve this, engineered fibrin-based hydrogels were covalently modified with VEGF₁₂₁. Methods allow the covalent incorporation of exogenous bioactive peptides by the transglutaminase activity of factor XIIIa into fibrin during coagulation. The ability of factor XIIIa to crosslink additional proteins within fibrin was employed to covalently incorporate VEGF₁₂₁. Other growth factors as described herein, particularly other isomers of VEGF, may be crosslinked to matrix materials such as fibrin by this method. Alternatively, the bioactive protein, preferably a growth factor, may be crosslinked to the matrix material by chemical means or the bioactive protein may be associated with the matrix by a non-covalent association.

Fibrin is a natural gel with several biomedical applications. Fibrin gel has been used as a sealant because of its ability to bind to many tissues and its natural role in wound healing. Some specific applications include use as a sealant for vascular graft attachment, heart valve attachment, bone positioning in fractures and tendon repair. Additionally, these gels have been used as drug delivery devices, and for neuronal regeneration. Although fibrin does provide a solid support for tissue regeneration and cell ingrowth, there are few active sequences in the monomer that directly enhance these processes.

The process by which fibrinogen is polymerized into fibrin has also been characterized. Initially, a protease cleaves the dimeric fibrinogen molecule at the two symmetric sites. There are several possible proteases than can cleave fibrinogen, including thrombin, reptilase, and protease III, and each one severs the protein at a different site. Each of these cleavage sites has been located. Once the fibrinogen is cleaved, a self-polymerization step occurs in which the fibrinogen monomers come together and form a non-covalently crosslinked polymer gel. This self-assembly happens because binding sites become exposed after protease cleavage occurs. Once they are exposed, these binding sites in the center of the molecule can bind to other sites on the fibrinogen chains, which are present at the ends of the peptide chains. In this manner, a polymer network is formed. Factor XIIIa, a transglutaminase activated from Factor XIII by thrombin proteolysis, may then covalently crosslink the polymer network. Other transglutaminases exist and may also be involved in covalent crosslinking and grafting to the fibrin network.

Once a crosslinked fibrin gel is formed, the subsequent degradation is tightly controlled. One of the key molecules in controlling the degradation of fibrin is a2-plasmin inhibitor. This molecule acts by crosslinking to the a-chain of fibrin through the action of Factor XIIIa. By attaching itself to the gel, a high concentration of inhibitor can be localized to the gel. The inhibitor then acts by preventing the binding of plasminogen to fibrin and inactivating plasmin. The a2-plasmin inhibitor contains a glutamine substrate.

The components required for making fibrin gels can be obtained in two ways. One method is to cryoprecipitate the fibrinogen from plasma, in which Factor XIII precipitates with the fibrinogen. The proteases are purified from plasma using similar methods. Another technique is to make recombinant forms of these proteins either in culture or with transgenic animals. The advantage of this is that the purity is much higher, and the concentrations of each of these components can be controlled.

In “Revascularization and Microcirculation of Freely Grafted Islets of Langerhans” published in World Journal of Surgery (April 2001, Volume 25, Number 4, 509-515) Michael D. Menger, Jun-ichiro Yamauchi, and Brigitte Vollmar of Institute for Clinical and Experimental Surgery, University of Saarland, D-66421 Homburg/Saar, Germany state that several experimental studies have demonstrated that the reestablishment of an appropriate microvascular supply is an essential prerequisite for successful pancreatic islet transplantation. Transplanted islets when injected freely into the liver show the first signs of angiogenesis (i.e., capillary sprout formation and protrusion) as early as 2 days after transplantation, and the entire vascularization process is completed after 10 to 14 days. A delay of 2 days in the initial vascularization of the islets causes some of the cells to die from lack of oxygen and nutrients, and vascularization not being fully completed for up to two weeks is unacceptable for maximum viability. The decreased viability and function requires the implantation of high quantities of islet to compensate for this loss. An increase in vascularization increases the quality of the islets and reduces the quantity needed for a curable dose.

Additionally, they state that cryopreservation and culture of the isolated islets before transplantation and hyperglycemia of the transplant recipient seem not to affect the vascularization process. Immunosuppressive drugs, such as cyclosporine A and 15-deoxyspergualin, do not inhibit or only slightly inhibit revascularization of syngeneic islets; however, they are not able to prevent completely xenograft-induced microvascular perfusion failure. In contrast, novel immunosuppressants (e.g., RS-61443) or dietary supplementation of the antioxidant vitamin E showed an almost complete prevention of microvascular graft rejection, including leukocyte recruitment and capillary perfusion failure. Novel strategies need to be developed to improve post-transplant islet function and should include concepts that accelerate the vascularization process and protect the newly formed microvasculature from rejection-mediated injury. The improvement of islet graft vascularization and the maintenance of adequate microvascular perfusion contributes to the increased success of pancreatic islet transplantation.

In “Development of a new method to induce angiogenesis at subcutaneous site of streptozotocin-induced diabetic rats for islet transplantation” (Cell Transplant. 2001;10(4-5):453-7) Gu Y, Tabata Y, Kawakami Y, Balamurugan A N, Hori H, Nagata N, Satake A, Cui W, Qi M, Misawa Y, Toma M, Miyamoto M, Nozawa M, and Inoue K. of Institute for Frontier Medical Sciences, Kyoto University, Japan state that subcutaneous implantation sites should be investigated for clinical islet transplantation. Even though there are several advantages to using a subcutaneous site, the poor blood supply is major factor in the failure of obtaining high islet survival. In this study, angiogenesis was induced in advance at the diabetic rats' subcutis for islet transplantation by implanting a polyethylene terepthalate (PET) mesh bag containing gelatin microspheres incorporating basic fibroblast growth factor (bFGF) (MS/bFGF) and a collagen sponge. The BFGF was incorporated into gelatin microspheres for controlled release of BFGF. Macroscopic and microscopic examinations revealed the formation of capillary network in and around the PET mesh bag containing MS/bFGF and collagen sponges 7 days after implantation when compared with control groups. When tissue hemoglobin level was also measured, a significantly high level of hemoglobin was observed compared with that of control groups. When allogeneic islets mixed with 5% agarose were transplanted into the prevascularized rat subcutis, normoglycemia was maintained for more than 40 days, while other control groups were ineffective. This study demonstrated that gelatin microspheres with bFGF and collagen sponges enabled the mesh to induce neovascularization even at the subcutaneous site of streptozotocin-induced diabetic rats, resulting in improved function of islet transplantation. However, the islets were only followed for 40 days, which is a very short period when considering a treatment for a life long disease, such as Diabetes. Additionally, the use of a mesh bag, which required implantation and removal for each treatment with islets is too invasive for a clinical treatment.

In “Low revascularization of experimentally transplanted human pancreatic islets” (J Clin Endocrinol Metab. 2002 December;87(12):5418-23) Carlsson P O, Palm F, and Mattsson G. of Department of Medical Cell Biology, Uppsala University, SE-751 23 Uppsala, Sweden, the authors observed that transplanted pancreatic islets are avascular immediately after implantation. The islets become revascularized, but it is uncertain whether the revascularization produces an adequate oxygenation of the transplanted islet tissue. They measured pO₂, blood flow and vascular density in mouse or human islets one month after transplantation to nude mice. The transplanted mouse and human islets both had a pO₂ 15-20% of that in endogenous mouse islets. Moreover, the vascular density of the transplanted islets was decreased compared with that of endogenous mouse and human islets. Graft blood perfusion was approximately 50% of renal cortex blood flow. A negative correlation was found between donor age and blood perfusion of the human islet grafts. A similar correlation was seen between donor age and the total vascular density of these grafts. They concluded, transplanted human islets had a markedly decreased vascular density and pO₂ compared with endogenous islets. This observation has potential implications for clinical islet transplantations, because poor vascular engraftment may significantly increase the number of islets needed to obtain insulin independence.

Currently, there are too few donors of pancreata to supply the necessary cell source to treat even a small number of diabetics. The problem will only increase if the available donor source is even further reduced by rejecting older patients due to poor vascularization of their islets.

In “Angiogenic capacity of endothelial cells in islets of Langerhans” (FASEB J. 2003 May;17(8):881-3) Linn T, Schneider K, Hammes H P, Preissner K T, Brandhorst H, Morgenstern E, Kiefer F, and Bretzel R G of Medical Clinic and Policlinic 3, Justus-Liebig University Giessen, Rodthohl 6, 35392 Giessen, Germany, the authors observed that the transplanted islets are disrupted from the surrounding blood vessels by the isolation procedure, with the grafted tissue being subject to ischemic damage. Survival of the transplanted islets is dependent on an effective revascularization. Perfusion studies suggest that newly formed microvessels supplying the graft with nutrients are exclusively rebuilt by the host. It is not known if the isolated islets contain endothelial cells (EC), which potentially participate in the revascularization process. They tried to detect immature EC in isolated islets by transformation with polyoma middle T antigen. Endothelioma cells were generated, implicating the presence of de-differentiated EC within isolated islets. When embedded in a fibrin gel, the islets developed cellular cords consisting of EC, whereas FGF-2 and VEGF stimulated the formation of cord-like structures. Also, they studied the presence of donor EC in islet grafts by using transgenic mice with an EC lineage-specific promotor-LacZ reporter construct (Tie-2LacZ). Following islet transplantation, Tie-2LacZ-positive EC of both donor and recipient were identified in the vicinity of or within the graft up to 3 wk after transplantation. EC and/or their progenitors with angiogenic capacity reside within isolated islets of different species, and their proliferative potential can be stimulated by various inducers. These graft-related endothelia persist after islet transplantation and are integrated within newly formed microvessels.

The observation of EC within the isolated islets demonstrates the need for a method to increase vascularization after transplantation. The quantity and function of the EC is variable and hard to measure. Therefore, an angiogenic growth combined with the encapsulated cells would give a predictable response and assure a higher survival of the implanted cells.

In “Induction of angiogenesis in omentum with vascular endothelial growth factor: influence on the viability of encapsulated rat pancreatic islets during transplantation” (J Vasc Res. 2003 July-August;40(4):359-67) Sigrist S, Mechine-Neuville A, Mandes K, Calenda V, Legeay G, Bellocq J P, Pinget M, and Kessler L. of Centre Europeen d'Etude du Diabete, Faculte de Medecine, Hopital de Hautepierre, Strasbourg, France, the authors observed that transplantation of pancreatic islets is a treatment for type 1 diabetes, but an insufficient blood supply could cause the loss of viable grafted islets. They investigated the influence of vascular endothelial growth factor (VEGF) on the angiogenesis of omentum during encapsulated islet allotransplantation and consequently on islet survival. Fifty rat islets, cultured for 24 h, were encapsulated in the presence or absence of human VEGF and implanted in the peritoneal cavity of rats (n=6). After 7, 14 and 28 days of implantation, encapsulation devices with surrounding omentum were removed. At each step in the study, there was a two-fold increase in the number of vessels in the presence of VEGF. In addition, VEGF increased the vessel diameter and the surface area of the angiogenic pedicle. Moreover, the presence of VEGF significantly decreased the distance between the devices and vessels (16.2±5.6 vs. 51.6±10.1 micron, p<0.001). Membrane surface analysis showed a decrease in macrophage adhesion in the presence of VEGF. Furthermore, islet structure and functionality was preserved in the presence of VEGF. Stimulation of angiogenesis of omentum induced by VEGF is associated with preservation of islet viability. Local delivery of VEGF proved to be a relevant approach to ameliorate the outcome of islet transplantation. The VEGF in this study was only present during the encapsulation step, rather than being released during the implantation of the islets. This method produces a variable and unpredictable quantity of VEGF at the implantation site, which in turn, produces a variable and unpredictable survival of the islets. Clinical treatments with VEGF must be quantifiable and predictable.

One preferred embodiment of the invention is related to compositions and methods of treating one or more diseases or disorders, such as neurologic (e.g., Parkinson's disease, Alzheimer's disease, Huntington's disease, Multiple Sclerosis, blindness, peripheral nerve injury, spinal cord injury, pain and addiction), cardiovascular (e.g., coronary artery, angiogenesis grafts, valves and small vessels), hepatic (e.g., acute liver failure, chronic liver failure, and genetic diseases effecting the liver), endocrine (e.g., diabetes, obesity, stress and adrenal, parathyroid, testicular and ovarian diseases), skin (e.g., chronic ulcers and diseases of the dermal and hair stem cells), hematopoietic (e.g., Factor VIII and erythropoietin), renal (acute renal failure, chronic renal failure), or immune (e.g., immune intolerance or auto-immune disease), in a subject in need of treatment comprising:

providing cells or tissue, such as pancreatic islets, hepatic tissue, endocrine tissues, skin cells, hematopoietic cells, bone marrow stem cells, renal tissues, muscle cells, neural cells, stem cells, embryonic stem cells, or organ specific progenitor cells, or genetically engineered cells to produce specific factors, or cells or tissue derived from such;

either using an unencapsulated said cell or;

enclosing said cells or tissue within at least one encapsulating material, such as a hydrogel, made of physically or chemically crosslinkable polymers, including polysaccharides such as alginate, agarose, chitosan, poly(amino acids), hyaluronic acid, chondroitin sulfate, dextran, dextran sulfate, heparin, heparin sulfate, heparan sulfate, gellan gum, xanthan gum, guar gum, water soluble cellulose derivatives, carrageenan, or proteins, such as gelatin, collagen, albumin, or water soluble synthetic polymers with ethylenically unsaturated groups or their derivatives, such as poly(methyl methacrylate) (PMMA), or poly(2-hydroxyethyl methacrylate) (PHEMA), poly(ethylene glycol) (PEG), poly(ethylene oxide) (PEO), poly(vinyl alcohol) (PVA), poly(vinylpyrrolidone) (PVP), poly(thyloxazoline) (PEOX); or a combination of the above, such as alginate mixed with PEG, or more hydrophobic or water insoluble polymers, such as poly(glycolic acid) (PGA), poly(lactic acid) (PLA), or their copolymers (PLA-GA), or polytetrafluoroethylene (PTFE); and

administering a therapeutically effective amount of said encapsulated cells or tissue to the subject in need of treatment via subcutaneous injection or implant, or directly into organs via either direct injection into the substance of the organ or injection through the vascular system of those organs.

Any angiogenic growth factor can be combined with a matrix to enhance the survival and functionality of the transplanted cells. Some of the angiogenic growth factors are Angiogenin, Angiotropin, Epidermal Growth Factor (EGF), Beta Fibroblast Growth Factor (β-FGF), Fibroblast Growth Factor-2 (FGF-2), Fibroblast growth factors (FGFs), Heparin-binding EGF-like growth factor, Hepatocyte growth factor (HGF), Insulin-Like Growth Factor I (IGF-I), Interferon-γ (IFN- γ), Interferon-g-inducible protein-10 (IP-10), Interleukin-8 (IL-8), Macrophage inflammatory protein-1 (MIP-1), Placental growth factor (PIGF), Platelet Derived Endothelial Cell Growth Factor, Platelet factor-4 (PF-4), Platelet-derived growth factor (PDGF), platelet-derived growth factor-BB (PDGF-BB), Pleiotrophin, Transforming Growth Factor α (TGF-α), Transforming Growth Factor β (TGF-β), and Vascular Endothelial Growth Factor (VEGF). This list is not meant to be limiting and additional factors would be known to one skilled in the art.

Organs maybe selected from, but not limited to, liver, spleen, kidney, lung, heart, brain, spinal cord, muscle, skin, and bone marrow. The subject in need of treatment may be selected from, but not limited to, mammals, such as humans, sub-human primates, cows, sheep, horses, swine, dogs, cats, and rabbits as well as other animals such as chickens, turkeys, or fish.

In a further embodiment of the invention, the encapsulated cell or tissue may be administered to a subject in need of treatment in combination with VEGF-fibrin glue and/or immunosuppressant and/or an anti-inflammatory agent. The immunosuppressant may be selected from, but not limited to cyclosporine, sirolimus, rapamycin, or tacrolimus. The anti-inflammatory agent may be selected from, but not limited to, aspirin, ibuprofen, steroids, and non-steroidal anti-inflammatory agents, and Cox-1 agents and Cox-2 agents. Preferably, the immunosuppressant and/or an anti-inflammatory agent is administered for six months following implantation or injection of the encapsulated cells or tissue. More preferably the immunosuppressant and/or an anti-inflammatory agent is administered for one month following implantation or injection of the encapsulated cells or tissue

In a preferred embodiment, encapsulated islets are implanted or injected subcutaneously or into liver or spleen. In one aspect of the invention, conformally coated islets are administered subcutaneously. In some embodiments, a double barrel syringe is used. Double barrel syringes have been commonly used to deliver materials such as fibrin or fibrin glues. Such devices are described in U.S. Pat. Nos. 4,359,049; 4,874,368; and 5,116,315 which are incorporated herein by reference. The double-barreled applicators keeps the fibrinogen-containing component separate from thrombin-containing component to avoid premature clot formation.

In an embodiment, the encapsulating material comprises a hydrogel that forms a sphere around at least one cell or tissue. In a further embodiment, the encapsulating material is an alginate microcapsule, which is conformally coated with another encapsulating material comprising acrylated PEG. In one embodiment, a cell or tissue may be encapsulated in a biocompatible alginate microcapsule, wherein the alginate is made biocompatible by coating the alginate in a biocompatible material, such as PEG or hyaluronic acid, purifying the alginate and/or removing the poly-lysine and replacing it with PEG.

Most preferably the disease to be treated is diabetes, the cells or tissue comprise insulin producing cells or tissue, or cells or tissue derived from pancreatic cells or tissue, or cells derived from progenitor or stem cells that are converted into insulin producing cells, and the encapsulated cells or tissue are administered to the subject in need of treatment via subcutaneous or liver injection or implant.

According to an embodiment of the invention, the microcapsules of encapsulated insulin-producing cells or tissue may have an average diameter of 10 μm to 1000 μm, preferably 100 μm to 600 μm, more preferably 150 μm to 500 μm, and most preferably 200 μm to 300 μm. In another embodiment, the invention relates to an insulin-producing cell or tissue encapsulated in microcapsules having a concentration of at least 2,000 IEQ (islet equivalents)/ml, preferably at least 9,000 IEQ/ml, and more preferably at least 200,000 IEQ/ml. In another embodiment of the invention, the volume of insulin-producing cells or tissue encapsulated in microcapsules administered per kilogram body mass of a subject may be 0.001 ml to 10 ml, preferably 0.01 ml to 7 ml, more preferably 0.05 ml to 2 ml. In a further embodiment of the invention, the ratio of microcapsule volume to insulin producing cell or tissue volume is less than 300 to 1, preferably less than 100 to 1, more preferably less than 50 to 1, and most preferably less than 20 to 1.

According to an embodiment of the invention, conformally coated insulin-producing cells or tissue may have an average membrane thickness of 1 to 400 μm, preferably 10 to 200 μm, and more preferably 20 to 50 μm. In a further embodiment, the invention relates to a conformally coated insulin-producing cell or tissue having a concentration of at least 10,000 IEQ/ml, preferably at least 70,000 IEQ/ml, more preferably at least 125,000 IEQ/ml, and most preferably at least 250,000 IEQ/ml. According to an embodiment of the invention the volume of the conformally coated insulin producing cell or tissue administered per kilogram body mass of a subject may be 0.01 to 7 ml, preferably 0.01 to 2 ml, and more preferably 0.04 to 0.5 ml. In another embodiment of the invention the ratio of conformal coating volume to insulin-producing cell or tissue volume is less than 13 to 1, preferably less than 8 to 1, more preferably less than 5 to 1, and most preferably less than 2.5 to 1.

According to an embodiment of the invention, the microcapsules of encapsulated cells or tissue may have an average diameter of 10 μm to 1000 μm, preferably 100 μm to 600 μm, more preferably 150 μm to 500 μm, and most preferably 200 μm to 300 μm. In a further embodiment of the invention, the ratio of microcapsule volume to insulin producing cell or tissue volume is less than 300 to 1, preferably less than 100 to 1, more preferably less than 50 to 1, and most preferably less than 20 to 1.

According to an embodiment of the invention, conformally coated cells or tissue may have an average membrane thickness of 1 to 400 μm, preferably 10 to 200 μm, and more preferably 20 to 50 μm. In another embodiment of the invention the ratio of conformal coating volume to cell or tissue volume is less than 13 to 1, preferably less than 8 to 1, more preferably less than 5 to 1, and most preferably less than 2.5 to 1.

An embodiment of the invention relates encapsulated cells or tissue where the cell density is at least about 100,000 cells/ml. Preferably, the encapsulated cell is conformally coated. More preferably, the cell is conformally coated with an encapsulating material comprising acrylated PEG. In a further embodiment, the invention is related to a method of treating diabetes in a subject comprising administering encapsulated islets where the cell density is at least about 6,000,000 cells/ml, preferably where the curative dose is less than about 2 ml per kilogram body mass of the subject.

Another embodiment of the invention is combining any angiogenic growth factor with a matrix to enhance the survival and functionality of the transplanted cells. Some of the angiogenic growth factors are Angiogenin, Angiotropin, Epidermal Growth Factor (EGF), Beta Fibroblast Growth Factor (β-FGF), Fibroblast Growth Factor-2 (FGF-2), Fibroblast growth factors (FGFs), Heparin-binding EGF-like growth factor, Hepatocyte growth factor (HGF), Insulin-Like Growth Factor I (IGF-I), Interferon-gamma (IFN-gamma), Interferon-g-inducible protein-10 (IP-10), Interleukin-8 (IL-8), Macrophage inflammatory protein-1 (MIP-1), Placental growth factor (PIGF), Platelet Derived Endothelial Cell Growth Factor, Platelet factor-4 (PF-4), Platelet-derived growth factor (PDGF), platelet-derived growth factor-BB (PDGF-BB), Pleiotrophin, Transforming Growth Factor α (TGF-α), Transforming Growth Factor β (TGF-β), and Vascular Endothelial Growth Factor (VEGF). This list is not meant to be limiting and additional factors would be known to one skilled in the art.

The invention is to a composition and method of administering or using a growth factor and conjugate, with transplanted cells, tissues or organs to enhance survival and function of the transplanted cells, tissues or organs. Some of these compositions or methods are:

A composition or method comprising a growth factor and conjugate, and encapsulating devices with a polyethylene glycol (PEG) coating having a molecular weight between 900 and 3,000 Daltons, wherein said composition has a cell density of at least about 100,000 cells/ml.

A composition or method comprising a growth factor and conjugate, wherein the encapsulating devices are microcapsules.

A composition or method comprising a growth factor and conjugate, wherein the microcapsules are conformally coated cell aggregates.

A composition or method comprising a growth factor and conjugate, wherein the cell aggregates are pancreatic islets.

A composition or method comprising a growth factor and conjugate, wherein the cell density is at least about 6,000,000 cells/ml.

A composition or method comprising a growth factor and conjugate, where the cell is selected from the group consisting of neurologic, cardiovascular, hepatic, endocrine, skin, hematopoietic, immune, neurosecretory, metabolic, systemic, and genetic.

A composition or method comprising a growth factor and conjugate, where the cell is selected from the group consisting of autologous, allogeneic, xenogeneic and genetically-modified.

A composition or method comprising a growth factor and conjugate, where the endocrine cell is an insulin-producing cell.

A therapeutically effective composition or method comprising a growth factor and conjugate, and a plurality of encapsulating devices having an average diameter of less than 400 μm, said encapsulating devices comprising encapsulated cells in an encapsulation material, wherein the composition comprises at least about 500,000 cells/ml.

A therapeutically effective composition comprising a growth factor and conjugate, wherein the average diameter of the encapsulating device is less than 300 micron.

A therapeutically effective composition comprising a growth factor and conjugate, and a plurality of encapsulating devices having an average diameter of less than 400 μm, said encapsulating devices comprising encapsulated cells in an encapsulation material, wherein the composition comprises a ratio of volume of encapsulating device to volume of cells of less than about 20:1.

A method of using the therapeutic composition comprising a growth factor and conjugate, and implanting said composition into an implantation site in an animal in need of treatment for a disease or disorder.

A method comprising a growth factor and conjugate, where the disease or disorder is selected from the group consisting of neurologic, cardiovascular, hepatic, endocrine, skin, hematopoietic, immune, neurosecretory, metabolic, systemic, and genetic.

A method comprising a growth factor and conjugate, wherein the endocrine disease is diabetes.

A method comprising a growth factor and conjugate, wherein the animal is from an Order of Subclass Theria selected from the group consisting of Artiodactyla, Carnivora, Cetacea, Perissodactyla, Primate, Proboscides, and Lagomorpha.

A method comprising a growth factor and conjugate, where the primate is a Human.

A method comprising a growth factor and conjugate, where the implanting is an injection.

A method comprising a growth factor and conjugate, where the implantation site is selected from the group consisting of subcutaneous, intraperitoneal, intramuscular, intraorgan, arterial/venous vascularity of an organ, cerebro-spinal fluid, and lymphatic fluid.

A method comprising a growth factor and conjugate, where the implantation site is subcutaneous.

A method comprising a growth factor and conjugate, and administering an immunosuppressant or anti-inflammatory agent.

A method comprising a growth factor and conjugate, where the immunosuppressant or anti-inflammatory agent is administered for less than 6 months.

A method of using a therapeutic composition comprising a growth factor and conjugate, and implanting said composition into an implantation site in an animal in need of treatment for a disease or disorder.

A method comprising a growth factor and conjugate, and further comprising implanting encapsulated islets in a subcutaneous implantation site.

A method comprising a growth factor and conjugate, and further comprising administering an immunosuppressant or anti-inflammatory agent.

A method comprising a growth factor and conjugate, where the biological material is selected from the group consisting of neurologic, cardiovascular, hepatic, endocrine, skin, hematopoietic, immune, neurosecretory, metabolic, systemic, and genetic.

A method comprising a growth factor and conjugate, where the biological material is from an animal of Subclass Theria of Class Mammalia.

Definitions:

As used in the present application, the following definitions apply:

| Term | Definition |
|---|---|
| Allografts | grafts between two or more individuals with different HLA or |
| | BLC immune antigen makeup at one or more loci (usually with |
| | reference to histocompatibility loci) within the same species |
| Athymic mice | a mouse with an incomplete immune system |
| Autograft | graft taken from one part of the body and returned to the same |
| | individual. |
| Biocompatibility | the ability to exist alongside living things without harming them |
| Cell aggregate | a collection of cells into a mass, unit, or an organelle that are |
| | held together be connecting substances, matrices, or structures |
| Clinically relevant and | encapsulating cell or tissue device must be of such a total |
| Clinical relevance | volume or size to be implantable in the least invasive or most |
| | physiologic site for function with the risk/benefit ratio below |
| | that of what the host with the disease or disorder faces with the |
| | current disease or disorder |
| Commercially relevant and | encapsulating cell device must be able to meet requirements |
| Commercial relevance | such as biocompatibility, permselectivity, encapsulated cell |
| | viability and function, size, cell retrieval or replacement, and |
| | therapeutic effect, in order for it to be produced on an ongoing |
| | basis for treatment of the disease process for which it has been |
| | designed within the acceptance as a product that is successful |
| | in the market place |
| Conformal Coating | a relatively thin polymer coating that conforms to the shape and |
| | size of the coated particle |
| C-peptide | the polypeptide chain in proinsulin linking the alpha and beta |
| | chains of active insulin. Insulin is initially synthesized in the |
| | form of proinsulin. There is one molecule of C-peptide for every |
| | molecule of insulin in the blood. C-peptide levels in the blood |
| | can be measured and used as an indicator of insulin production |
| | when exogenous insulin (from injection) is present and mixed |
| | with endogenous insulin (produced by the body). The C- |
| | peptide test can also be used to help assess if high blood |
| | glucose is due to reduced insulin production or to reduced |
| | glucose intake by the cells. Type 1 diabetics have little or no C- |
| | peptide in the blood, while Type 2 diabetics can have increased |
| | or normal C-peptide levels early in the progress of the |
| | syndrome and reduced C-peptide levels later in the syndrome. |
| | The concentration of C-peptide in non-diabetics is 0.5-3.0 ng/ml. |
| Cynomolgus primate | crab-eating macaque, Macaca fascicularis, is native to Southeast |
| | Asia. |
| Diabetes | a variable disorder of carbohydrate metabolism caused by a |
| | combination of hereditary and environmental factors and |
| | usually characterized by inadequate secretion or utilization of |
| | insulin, by excessive urine production, by excessive amounts of |
| | sugar in the blood and urine, and by thirst, hunger, and loss of |
| | weight |
| DTZ | a dye that binds to the zinc within insulin granules |
| (diphenylthiocarbazone) | |
| HbA1c test - [equivalent | test used to assess long-term glucose control in diabetes. |
| to Hemoglobin A1C; | Alternative names for this test include glycosylated hemoglobin |
| Glycated hemoglobin] | or Hgb, hemoglobin glycated or glycosylated protein, and |
| | fructosamine. HbA1c refers to total glycosylated hemoglobin |
| | present in erythrocytes. Due to the fact that glucose stays |
| | attached for the life of the cell (about 3 months), the test shows |
| | what the person's average blood glucose level over a period of |
| | 4-8 weeks in humans. This is a more appropriate test for |
| | monitoring a patient who is capable of maintaining long-term, |
| | stable control. Test results are expressed as a percentage, with |
| | 4 to 6% considered normal in humans. The HbA1c “big picture” |
| | complements the day to day “snapshots” obtained from the |
| | self-monitoring of blood glucose (mg/dL), and the two tests can |
| | be related with the conversion equation: HbA1c = (Plasma |
| | Blood Glucose + 77.3)/35.6. Glycated protein in |
| | serum/plasma assesses glycemic control over a period of 1-2 |
| | weeks. |
| IP (Intraperitoneal) | within the peritoneal cavity, the area that contains the |
| | abdominal organs. |
| IEQ (Islet equivalent) | defination based on both insulin content and morphology/size. |
| | An insulin granule binding dye, such as diphenylthiocarbazone |
| | (DTZ) is commonly used to identify beta cells. Since beta cells |
| | are only one of several other cell types needed to constitute an |
| | islet, a morphological assessment, based upon a mean diameter |
| | of 150 μm, is used in addition to staining by DTZ, to define an |
| | islet equivalent. |
| Maturity Onset Diabetes | a form of diabetes characterized by early age of onset (usually |
| of the Young (MODY) | less than 25 years of age), autosomal dominant inheritance |
| | (that is, it is inherited by 50% of a parent's children) with |
| | diabetes in at least 2 generations of the patient's family. During |
| | the early stages, MODY diabetes that can often be controlled |
| | with meal planning or diabetes pills. It differs from Type 2 |
| | diabetes in that patients have a defect in insulin secretion or |
| | glucose metabolism, and are not resistant to insulin. MODY |
| | accounts for about 2% of diabetes worldwide. So far, six genes |
| | have been found that cause MODY; however, not all MODY |
| | patients have one of these genes. MODY is useful for studying |
| | diabetes genes because it runs in families and researchers |
| | obtain useful information on insulin production and regulation |
| | by the pancreas. |
| MDCK cells - (Madin- | epithelial-like cell line established from normal kidney of dog, |
| Darby canine kidney) | susceptible for many viral species. |
| Microcapsules | small particles that contain an active agent or core material |
| | surrounded by a coating or shell. |
| Modified Growth Factor | a growth factor with an attached amino acid sequence that |
| | comprises a substrate domain for an enzyme that covalently |
| | binds the growth factor to the matrix. |
| Oral Glucose Tolerance | a screening test for diabetes that involves testing an individual's |
| Testing (OGTT) | plasma glucose level after he drinks a solution containing 75 |
| | grams of glucose. Currently, a person is diagnosed with |
| | diabetes if his plasma glucose level is 200 mg/dL or higher two |
| | hours after ingesting the glucose. Those with a plasma glucose |
| | level less than 200 mg/dL but greater than or equal to 140 mg/dL |
| | are diagnosed with a condition called impaired glucose |
| | tolerance. People with this condition have trouble metabolizing |
| | glucose, but the problem is not considered severe enough to |
| | classify them as diabetic. Individuals with impaired glucose |
| | tolerance are at a slightly elevated risk for developing high |
| | blood pressure, blood lipid disorders, and Type 2 diabetes. |
| Permselectivity | preferential permeation of certain ionic species through a |
| | membrane. |
| PoERV (porcine | an endogenous retrovirus exists as part of the DNA in all |
| endogenous retrovirus) | mammals and is passed down to offspring over successive |
| | generations. |
| Postprandial | occurring after a meal |
| Proinsulin | a protein made by the pancreas beta cells, which is cleaved into |
| | 3 units - C-peptide, alpha chain and beta chain. The alpha and |
| | beta chains are the functional units of insulin. |
| Streptozotocin | an antibiotic, C₈H₁₅N₃O₇, produced by an actinomycete |
| | (Streptomyces achromogenes) and active against tumors but |
| | damaging to insulin-producing cells and now regarded as a |
| | carcinogen. |
| Therapeutically effective | amount of a therapeutic agent produced by cells or tissue |
| amount | which, when administered to a subject in need thereof, is |
| | sufficient to effect treatment for a disease or disorder, or to |
| | effectively change the growth rate or alter the condition of an |
| | animal. The amount of encapsulated cells or tissue |
| | corresponding to a “therapeutically effective amount” will vary |
| | depending upon factors such as the disease condition and the |
| | severity thereof, the identity of the subject in need thereof, and |
| | the type of therapeutic agent delivered by the cells or tissue for |
| | the disease or disorder, but can nevertheless be readily |
| | determined by one of skill in the art. |
| Treating and Treatment | to alleviate a disease or disorder in a subject, such as a human, |
| | by the dosage of encapsulated cells or tissue to the subject in |
| | need of treatment via subcutaneous injection or implant, or |
| | directly into organs via either direct injection into the substance |
| | of the organ or injection through the vascular system of those |
| | organs and includes: (a) prophylactic treatment in a subject, |
| | particularly when the subject is found to be predisposed to |
| | having the disease or disorder but not yet diagnosed as having |
| | it; (b) inhibiting the disease or disorder; and/or(c) eliminating, in |
| | whole or in part, the disease or disorder; and/or(d) improving |
| | the subject's health and well-being. |
| Type 1 diabetes (also | diabetes mellitus is an autoimmune disease requiring a genetic |
| insulin-dependent | predisposition combined with an environment agent to activate |
| diabetes, insulin- | the autoimmune process that usually develops during childhood |
| dependent diabetes | or adolescence and is characterized by a severe deficiency in |
| mellitus) | insulin secretion resulting from atrophy of the islets of |
| | Langerhans, and causing hyperglycemia and a marked tendency |
| | towards ketoacidosis. |
| Type 2 diabetes (also | a common form of diabetes mellitus that develops especially in |
| non-insulin-dependent | adults and most often in obese individuals and that is |
| diabetes, non-insulin- | characterized by hyperglycemia resulting from impaired insulin |
| dependent diabetes | utilization or insulin resistance coupled with the body's inability |
| mellitus) | to compensate with increased insulin production. |
| Xenografts | a surgical graft of tissue from one species onto or into |
| | individuals of unlike species, genus or family. Also known as a |
| | heteroplastic graft. |

Detailed Description

The present invention relates to methods of treating a disease or disorder by implanting encapsulated biological material enmeshed in a matrix with a growth factor and conjugate into patients in need of treatment. Diabetes is of particular interest because a method is needed to prevent complications related to the lack of good glycemic control in insulin-requiring diabetics. Specifically, PEG conformally coated islet allografts enmeshed in a matrix with a growth factor and conjugate in diabetic primates have been successfully implanted in the subcutaneous site by injection and achieved relatively normal blood glucose values post-implant. The current complications of clinical islet transplantation, and the significant risks and discomfort of continuous immunosuppression can be eliminated by applying the methods described herein to patients with insulin-requiring diabetes. In addition, encapsulated islet implants are expected to protect these insulin-requiring diabetic patients and prevent them from developing the complications from diabetes related to inadequate glycemic control in spite of exogenous insulin therapy.

Methods according to the present invention may provide therapeutic effects for a variety of diseases and disorders, in addition to diabetes, in which critical cell-based products lost by disease or disorder may be replaced through implantation of cells or tissue into the body. A preferred embodiment of the invention is the use of human insulin-producing cells from the pancreas, or cells derived from human insulin-producing cells from the pancreas, that are encapsulated as cell clusters for implantation into the subcutaneous site of insulin-requiring patients. Treatment of disease via encapsulated biological materials requires that the encapsulated material be coated with a biocompatible coating, such that the immune system of the patient being treated does not destroy the material before a therapeutic effect can be realized.

Permselectivity of the coating is a factor in the effectiveness of such treatments, because this regulates the availability of nutrients to the cells or tissue, and plays a role in preventing rejection of the biological materials. Permselectivity of the coating affects the nutrition available to the encapsulated cell or tissue, as well as the function of the cell or tissue. Permselectivity can be controlled by varying the components of the biocompatible coating or by varying how the components are used to make the cell coating. Treatment via injection of encapsulated biological materials according to the present invention provides a stable and safe method of treatment. Size of the implant and the site of implantation, as well as replenishment and/or replacement of the encapsulated materials is also a consideration of the methods described herein. These methods provide a treatment that has a wide range of applications in the treatment of disease at various sites of implantation, while avoiding complications associated with other treatment methods.

The conformal coatings described herein can be produced with different pore sizes that can be produced to limit access to the cells by proteins of widely varying molecular weights, including the exclusion of antibodies. This control allows for survival and maintained function of the encapsulated materials, while excluding components of the host immune system. The appropriate pore size of the conformal coating may be determined by routine experimentation for each cell or tissue type and the disease or disorder to be treated. The conformal coatings described herein provide a small encapsulated cell product with a minimal volume of the coating material, thus allowing the coated materials to be implanted into various sites of the body, including direct injection into the liver, spleen, muscle, or other organs, injection via vascular access to any organ, injection into the abdominal cavity, and implantation into a subcutaneous site.

An important factor for successful encapsulated cell therapy is that the permselective coating used to encapsulate the cells must be inert in terms of causing inflammatory reactions in the host. Most previous encapsulating materials were not completely biocompatible. With some devices, not making a large scar is sufficient. However, when using the coating for permselective protection between the encapsulated cells and the host immune system, there cannot be any non-specific inflammatory reaction to the hosts complement system or to macrophages. If this occurs, then the inflammatory and/or immune reaction is sufficient to release cytokines that readily cross the membrane and can cause the loss of the encapsulated cells. Most encapsulation technologies for islets, which have had difficulties in working appropriately, had non-specific inflammatory reactions due to biocompatibility reactions to the coating materials.

Problems such as chronic inflammation are significantly reduced due to the lack of host reaction to the biocompatible conformal coatings used to encapsulate cells and tissues used in the methods described herein. The components used to produce the conformal coating described herein have been shown to be completely biocompatible when injected into animals, such as, rodents, dogs, pigs, and primates.

We discovered that biocompatibility of hydrogels synthesized from highly acrylated PEG was exceptionally good, and much better than that shown with moderately acrylated PEG hydrogels. The highly acrylated PEGs were either obtained commercially, or homemade by acrylating corresponding PEGs. Hydrogels with highly acrylated PEGs were conformally coated on the surface of alginate microbeads using an interfacial photopolymerization technology. This discovery also can be extended to other biomedical, biotechnological and pharmaceutical areas where biocompatibility of the devices or formulations is of concern.

Some PEG conformal coatings described herein are biodegradable over time, thus allowing the body to safely break down the materials over the course of time and avoiding the need to retrieve the encapsulated materials, which is required by other treatments. Replacement of cells can be done whenever the previous dose of encapsulated materials has begun to lose function. Encapsulated islets may be expected to last two to five years or longer. In the case of subcutaneous injections, replacement of the encapsulated materials may simply be done via another percutaneous injection of new materials into the patient at a different site prior to loss of the previous dose. In the case of encapsulated islets, this replacement can be done prior to loss of function in the first dose of islets, without fear of low glucose values, because the encapsulated islets autoregulate themselves to prevent hypoglycemia. Different implant timing may have to be determined for treating diseases and disorders using cells or tissues do not autoregulate the release of their product.

A factor in producing encapsulated cell products is the cell source. Cells may be stem cells, primary cells, expanded cells, differentiated cells, cell lines, or genetically engineered cells. In the case of human islets, primary islets may be isolated from cadaver-donated pancreases; however, the number of human pancreata available for isolating islets is very limited. Alternative cell sources may be used to provide cells for encapsulation and injection.

One alternative source of cells, particularly insulin-producing cells, is embryonic stem cells. Human embryonic stem cells come from the very early fetus. They are only available when grown from frozen, fertilized human eggs collected from couples that have successfully undergone in vitro fertilization and no longer want to keep these fertilized eggs for future children. Embryonic stem cells have the ability to grow indefinitely, potentially avoiding the need for the mass of tissues required for transplantation. There are a series of steps required to differentiate these embryonic stem cells into insulin producing cells with clinical relevancy. A few studies have shown both mouse and human embryonic stem cells can produce insulin when treated under tissue culture with a variety of factors. Insulin-producing cells developed from embryonic stem cells may be an acceptable cell source for transplantation, and encapsulated cell or tissue implantation.

Cell Sourcing

Additional cell sources, organ specific progenitor cells from the brain, liver, and the intestine, have been shown to produce insulin. In order to produce insulin, each of these organ specific progenitor cells has undergone tissue culture treatments with a variety of growth and differentiation factors. Additional organ specific progenitor cells from many other organs such as bone marrow, kidney, spleen, muscle, bone, cartilage, blood vessels, and other endocrine organs may also be useful in providing insulin producing cells.

Pancreatic progenitor cells may be used according to the methods of the invention. The pancreas seems to have organ specific stem cells that can produce the three pancreatic cell types in the body under normal and repair conditions. It is believed the islet cells bud off from the duct cells to form the discrete islets. The insulin producing beta cells, as well as the other hormone producing cells, may form directly from differentiating duct cells or may form from pancreatic progenitor cells located amongst the duct cells. These pancreatic progenitor cells may be used to provide insulin-producing cells for encapsulation and implantation according to the methods described herein.

There has been a great deal of research on genetically inserting genes into non-insulin producing cells to make them produce insulin. Genetically engineered cells capable of insulin production may also be used for encapsulation and implantation according to the methods described herein.

The use of pig cells has been considered as a source of islet cells for implantation in patients with diabetes. Over 90 million pigs are raised per year for meat production in the USA alone. Therefore, the number of islets to treat the millions of patients with insulin-requiring diabetes is readily available through large scale processing of adult pig pancreata into purified pig islets for encapsulation. One consideration limiting this choice is the recognition that pigs harbor an endogenous retrovirus (PoERV). There have been efforts to eliminate PoERV from strains of pigs. Virus-free pig xenograft islets may be readily encapsulated and available as a preferred cell source for the treatment of human diabetes.

Alternative xenograft sources for human implantation may be obtained from primary cells of species other than pigs. These other species could be agriculturally relevant animals such as beef, sheep, and even fish. With the ability to expand and differentiate insulin producing cells from pancreatic sources or other stem or progenitor cells, one can envision using insulin-producing cells from many other xenogeneic sources such as primates, rodents, rabbits, fish, marsupials, ungulates and others.

Disease Treatment

Diabetes and other diseases in which a local or circulating factor is deficient or absent can be treated according to the methods described herein. Encapsulated cell therapy may be applied in the treatment of neurologic, cardiovascular, hepatic, endocrine, skin, hematopoietic, and immune disorders and diseases. Neurologic diseases and injuries, such as Parkinson's disease, Alzheimer's disease, Huntington's disease, multiple sclerosis, blindness, spinal cord injury, peripheral nerve injury, pain and addiction may be treated by encapsulating cells that are capable of releasing local and/or circulating factors needed to treat these problems. Cardiovascular tissue, such as the coronary artery, as well as angiogenic growth factor releasing cells used for restoring vascular supply to ischemic cardiac muscle, valves and small vessels may be treated. Acute liver failure, chronic live failure, and genetic diseases affecting the liver may be treated. Endocrine disorders and diseases, such as diabetes, obesity, stress and adrenal, parathyroid, testicular and ovarian diseases may be treated. Skin problems, such as chronic ulcers, and diseases of the dermal and hair stem cells can be treated. Hematopoietic factors such as Factor VIII and erythropoietin may be regulated or controlled by administering cells capable of stimulating a hematopoietic response in a patient. Encapsulated biological materials may also be useful in the production of bone marrow stem cells. Encapsulated materials, such as, antigens from primary cells or genetically engineered cells, may be useful in producing immune tolerance or preventing autoimmune disease. In addition, these materials may be used in vaccines.

### EXAMPLES

### Example 1

Subcutaneous Implant of Encapsulated Islets in Baboons

Surgical Procedures: Baboon pancreata were removed from donors, cannulated, and flushed with pancreas preservation solution and then shipped to Novocell for islet preparation and encapsulation. They were subsequently cultured, shipped to the holding facility for implantation, and then prepared for surgical implantation by suspension in culture medium, using similar protocols as are proposed for human islet preparation. The baboons were anesthetized, and a 16-gauge catheter was placed into the subcutaneous site of the anterior abdomen. A trochar was inserted through the implanted catheter to create a “fan shaped” area of 1-10 subcutaneous tracts (˜1″-5″ each in length) under the skin of the abdomen. The test material (in 1 to 10 ml volume) was gently suspended, pulled into a syringe, and deposited along the subcutaneous tracts (or “pockets”) with an even pattern of deposition throughout the pockets. The needle insertion site was closed with a purse string suture to prevent any leakage from the insertion site. This resulted in long, low-lying areas of test material and buffer. The liquid portion was quickly resorbed and left a slightly granular surface texture. Several sites were used for the complete implantation procedure. The area was tattooed to mark the injection site location. No local reaction was noted indicating inflammation.

Drug Treatments: Cyclosporine (at a sub-immunosuppressive dosage with 24-hour trough levels from 25-200 ng/ml) was administered on days −7 through +30 post-implant. Cyclosporine was administered to prevent collateral loss of encapsulated islets due to immediate focal allograft immune response to some weakly encapsulated islets in the implant. Additionally, to mimic clinical concomitant medications, metformin was administered starting on day +1 and throughout the duration of the study. The dose of coated islets delivered at least 4 weeks post streptozotocin administration to induce diabetes was approximately 10-500 K IEQ/kg body weight. The difference between the effective islet dose used during our studies and the dose used in current human studies (15K IEQ/kg) was likely due to the implant site (subcutaneous vs. portal vein) and loss of islets following implantation.

Monitoring: The aim of the in-life monitoring was to provide comprehensive assessment of information needed to track diabetic management and implant activity, as well as standard indicators of local tolerance and global indicators of overall health/safety assessment. The groups were monitored during the pre-diabetic period (baseline), during the diabetic period, and post-implant. Pre-diabetic, diabetic and monthly post-implant measurements included OGTT and AST (Arginine Stimulation test) (with blood glucose, insulin and c-peptide assays), and hemoglobin A1c. Daily monitoring of diabetic and post-implant periods included blood glucose (fasting, 2 hour post prandial and pre-dinner), urinary glucose and ketones (morning fasting and pre-dinner), food intake (grams of carbohydrate, fat and protein), amount of insulin injected (diabetes management) and other medication doses. Weekly measurements included body weight and clinical observations.

Necropsy: Histopathologic examination of the subcutaneous implant site and a non-implanted control site were performed using hematoxylin and eosin (H&E) staining and immunohistochemistry staining (insulin, glucagon, VEGF receptor, inflammatory macrophages, dendritic macrophages, and activated lymphocytes, and lymphocytes CD4 and CD8). Histopathologic examination of all standard organs and tissues were conducted using H&E staining and evaluated by a board-certified veterinary pathologist. Immunohistological staining of the pancreas was conducted to evaluate the presence of insulin and glucagon.

### Example 2

Encapsulated Islet Allograft without VEGF in Streptozotocin Diabetic Baboons

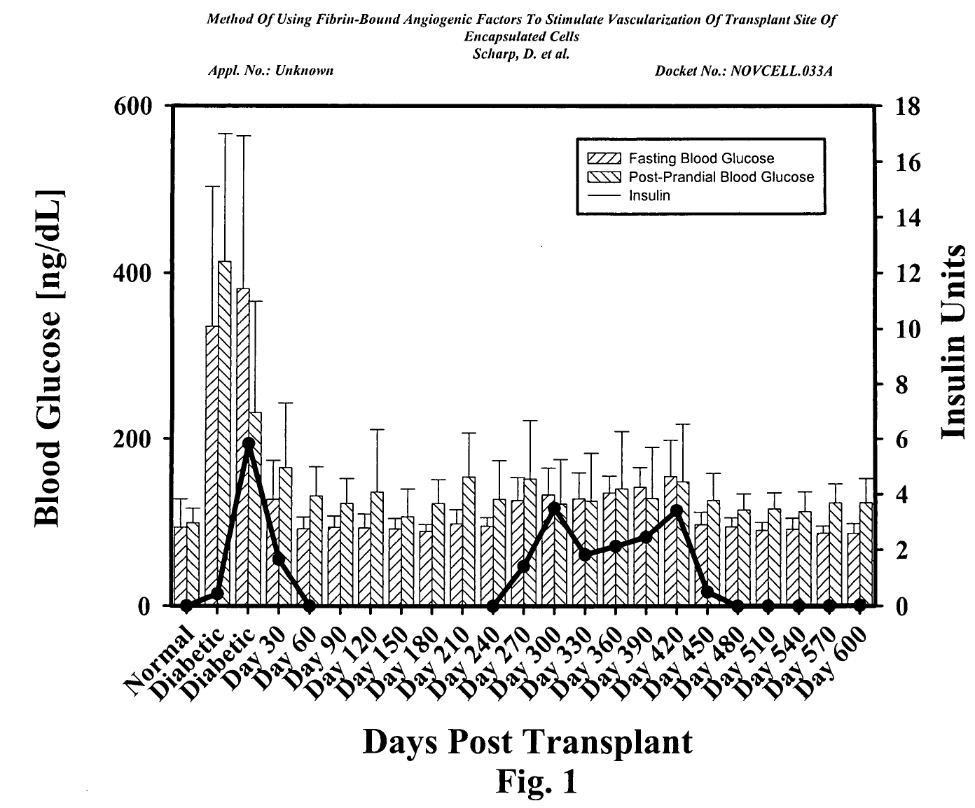

FIG. 1 shows the results of a diabetic baboon implanted with encapsulated islet allografts. This diabetic baboon recipient showed the ability to achieve insulin independence within 17 days after subcutaneous implantation of encapsulated islet allografts. This was in contrast to Cynomolgus primate diabetics where insulin independence was only achieved 30 days after islet implantation.

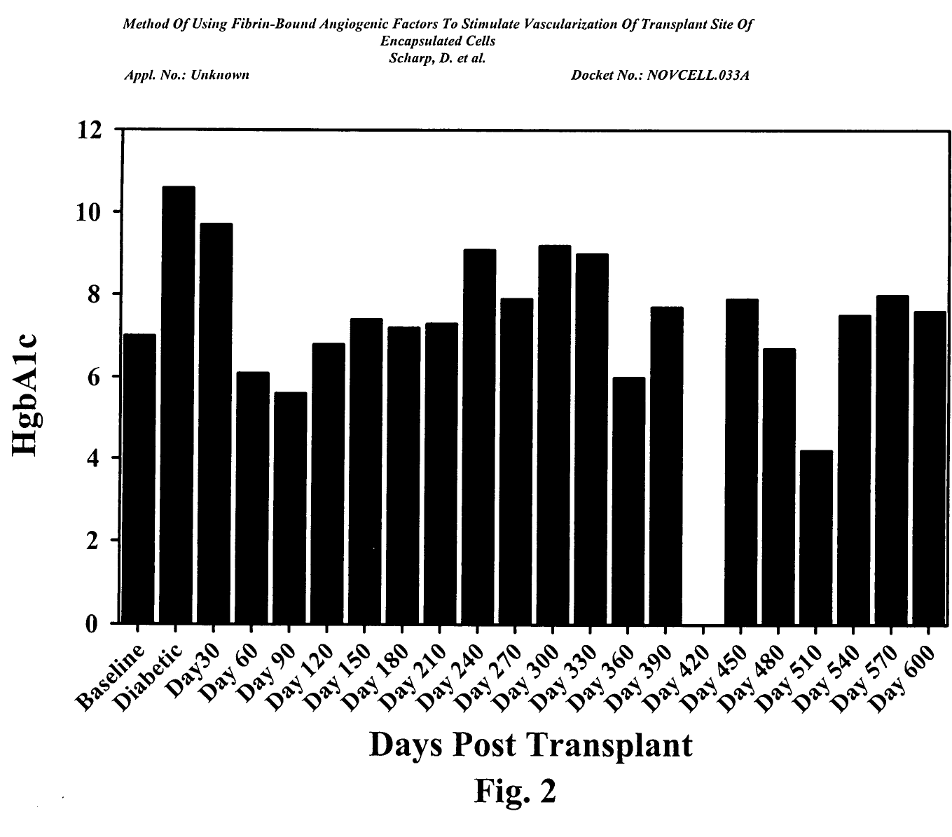

The baboon diabetic model used IM injection of cyclosporine for administration of the drug to these large animals. This eliminated the variances in the 24-hour trough levels. FIG. 2 shows this recipient achieved normal Hemoglobin A1c levels by 60 days post-implant and remained in the normal level through 210 days without insulin. There were slight increases followed by decrease between day 210 and day 360. The animal returned to partial graft function at day 240 requiring low doses of insulin.

At day 420, the animal received a second subcutaneous implantation of encapsulated islet allografts. Once again the diabetic baboon recipient achieved insulin independence after subcutaneous implantation of encapsulated islet allografts.

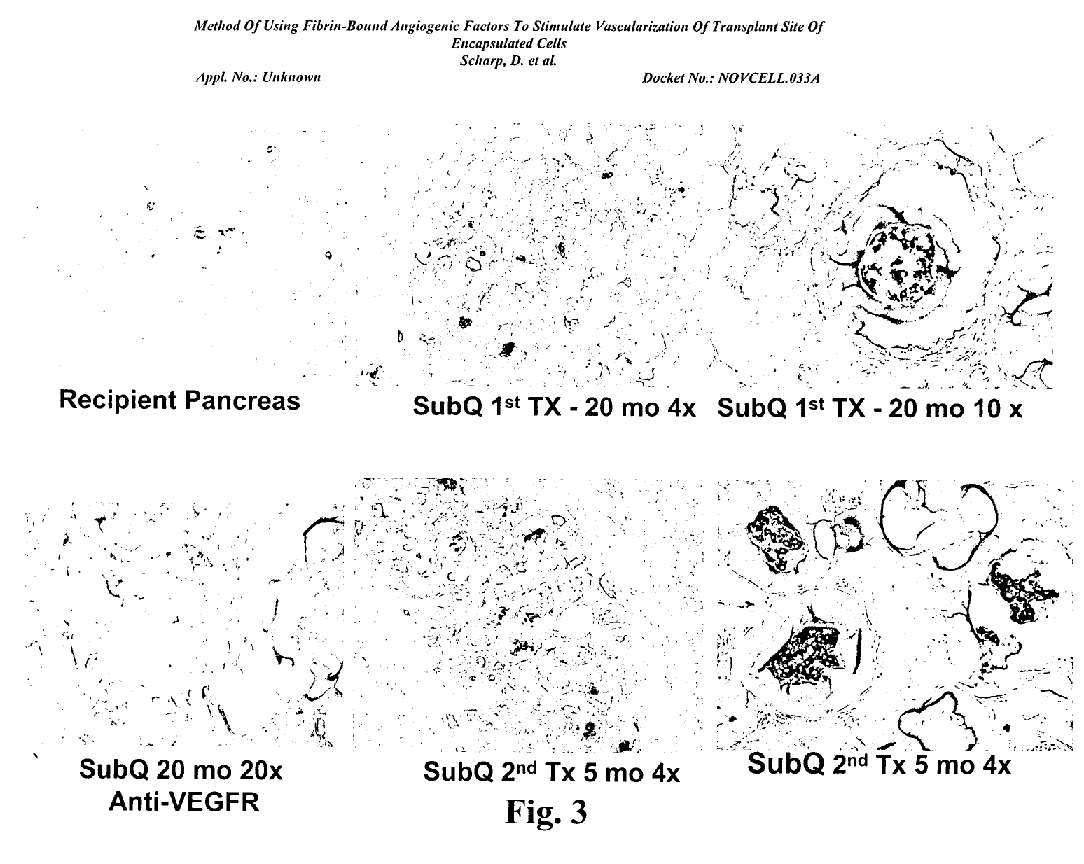

FIG. 3 shows the histology of the encapsulated islet allografts from both implantations, 20 months and 5 months. The cells are viable and there is no inflammatory response to the implantation.

Results of the OGTT and AST demonstrated significant C-peptide release following all time points after implantation. The normal response showed a peak of C-peptide at the 30-minute period with the values decreasing thereafter resulting in normal glucose values at all periods. During the diabetic period, the glucose values continued to rise throughout the test with very low levels of C-peptide that were not responsive to the glucose challenge. Following the implantation, there were increased levels of C-peptide to glucose challenge but the response was delayed with peaks occurring at 60 and 90 minutes post-challenge. Examining the glucose values, the 30 and 60-minute values were higher than normal as expected from the delay in C-peptide levels. However, by 90 and 120 minutes, the glucose values returned close to normal. At this time, it is not known whether this delay in C-peptide production was due to the subcutaneous implantation site or to the encapsulation of the islets. These results were very analogous with implanted islets in the portal vein of human diabetics under immunosuppression.

A second baboon was implanted in the subcutaneous site with low dose cyclosporine. A lowering of the glucose values occurred while maintaining nearly the same insulin requirement. The insulin requirement slowly dropped at day 100, but slowly rose until nearly day 200, while the glucose values remained lowered. Examination of the hemoglobin A1c values showed that partial function was clearly achieved by lowering the levels significantly from 12% to 8.0% by day 90 and to normal levels at day 120. The values slowly rose to the 8% level where they remained at 180 days. These values showed a partial function that was compatible with those being achieved with islet transplant recipients that do not achieve insulin independence but which maintain near normal levels of hemoglobin A1c levels post-implant.

Examination of the OGTT and AST results showed lower C-peptide values and higher glucose values throughout the post-implant period compared to the first recipient. Yet, the C-peptide levels were significantly higher than the values obtained during the time of diabetes.

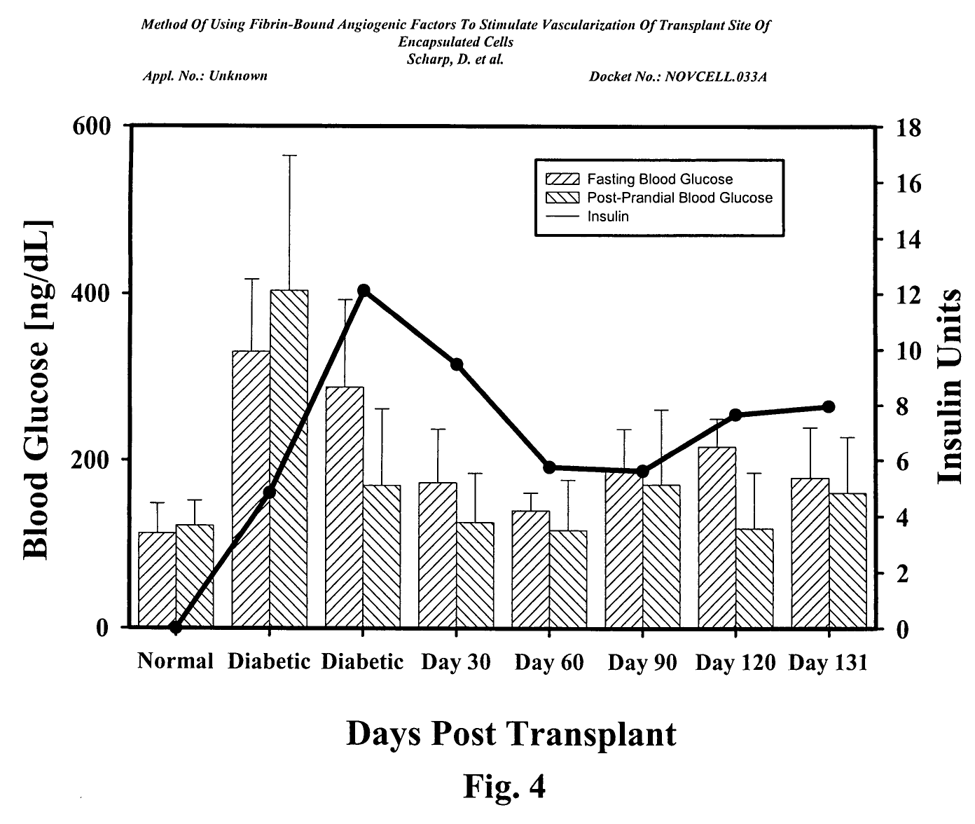

A third recipient also received encapsulated islet allografts in the subcutaneous site with low dose cyclosporine (FIG. 4). This recipient demonstrated a partial response following implantation with over a 50% decrease in both the glucose and insulin values compared to the diabetic period. These values were maintained until 140 days post-implantation.

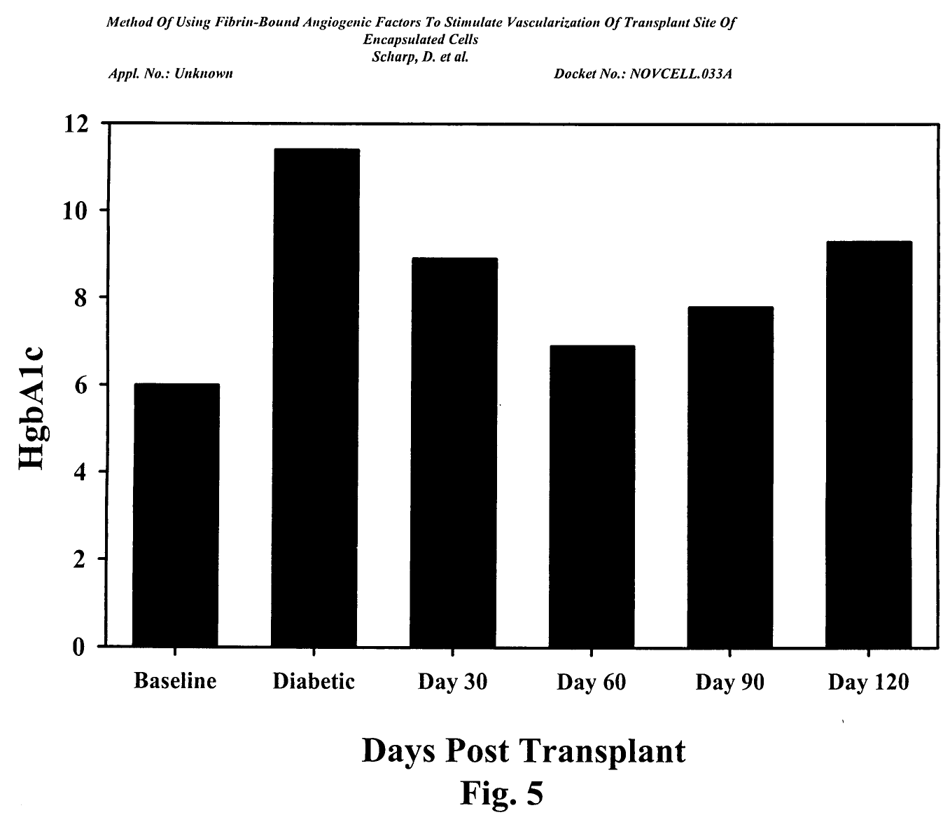

The hemoglobin A1c values (FIG. 5) for this recipient showed that it reached a normal range by 60 days post-implant, but rose to diabetic levels by 120 days. This again demonstrated a partial islet function for 90 days with reduced responsiveness after that time.

The responses to both OGTT and AST were similar to those observed for the previous partially functioning recipient with elevated C-peptide values post-implant that peak in the 60 to 90 minute period.

It was important to understand how these results (one normal islet transplant recipient and two partial recipients) compared with those from implantation into the portal vein of a diabetic recipient. A pre-study baboon recipient, which had been the first to receive encapsulated islets in the subcutaneous site, was used to accomplish this task. Two marginal implants were performed at day 0 and day 110, with only transient improvements, as expected. On day 240 after the first implant, an intraportal vein injection of encapsulated islet allografts was made without any significant rise in portal venous pressure or any change in liver function tests. There was a dramatic response to the implant with a greater than 50% drop in the insulin requirement within a few days. After the cyclosporine was stopped at 30 days post-implant, this recipient came off insulin treatment with normal glucose levels up to 290 days post-implant. Examining the hemoglobin A1c values for this recipient, a reduction followed the marginal subcutaneous implants, but not to normal levels. The only value following the portal vein implant was taken at the 30-day post-implant period, which was too early to see the expected improvement. Hemoglobin A1c levels lag behind the clinical results by approximately 30 days in the baboon.

Following the marginal subcutaneous islet implants, there were low levels of C-peptide remaining that were higher than the diabetic values. Although the hemoglobin A1c levels were reduced, the implant did not cause normal levels. There was a marked drop in the glucose response that was associated with a significant rise in C-peptide. The significant improvement from the portal vein implant suggested the potential of enhancing the subcutaneous site to obtain improved results.

### Example 3

Subcutaneous Site Enhancement by Implanting Encapsulated Islet Allografts within Fibrin Glue Conjugated with VEGF

There was a continual finding of new blood vessel formation surrounding the PEG encapsulated islets in the subcutaneous site several weeks following implant. The subcutaneous site was enhanced by an angiogenic growth factor at the time of implant to increase the survival of the encapsulated islets during the first few weeks post-implant. This approach reduced the number of isolated islets required to eliminate diabetes.

A new angiogenic reagent has been developed that is used for wound healing. A human fibrin glue product is used as a base for adding VEGF. The conjugated VEGF breaks down at the same time as the fibrin clot breaks down in the body. A slow release of VEGF at the site causes the development of normal capillaries.

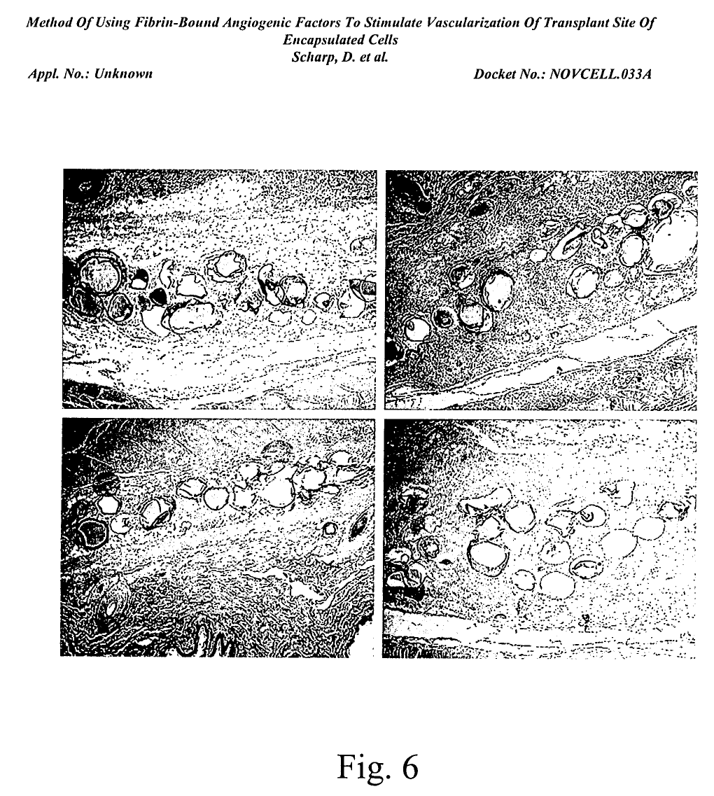

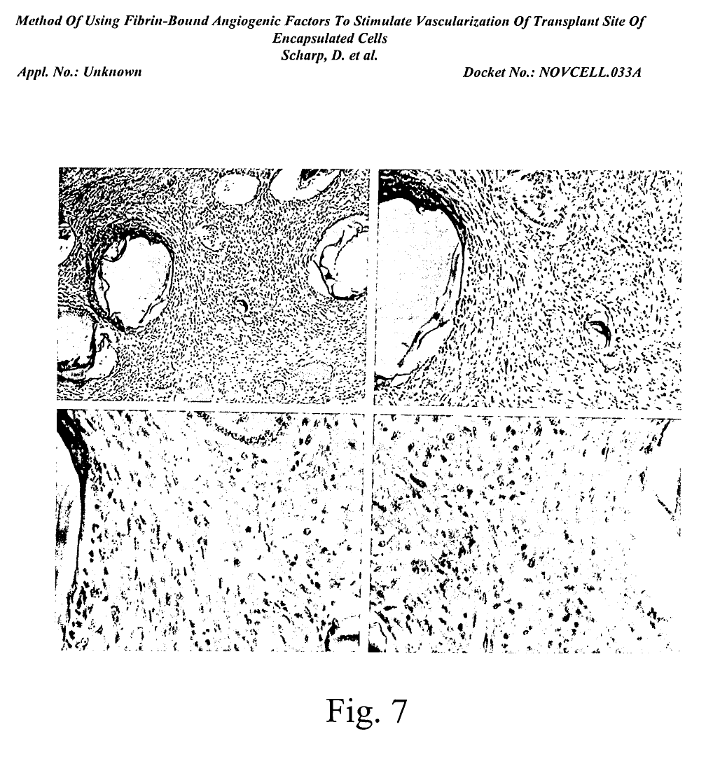

These results were confirmed by transplanting empty PEG beads, as well as, PEG conformally coated islets in diabetic mice and non-diabetic primates (FIGS. 6 and 7).

Two diabetic baboons were implanted using the VEGF conjugated fibrin. The first baboon received three doses of streptozotocin to achieve a severe diabetic state and then stabilized with 8-10 units of insulin per day until injected with encapsulated cells. Then, it received a subcutaneous implant of islets entrapped in a fibrin clot with slow release VEGF. Injecting this combination was not difficult in the subcutaneous site and resulted in clot formation that was palpable for 10 to 14 days.

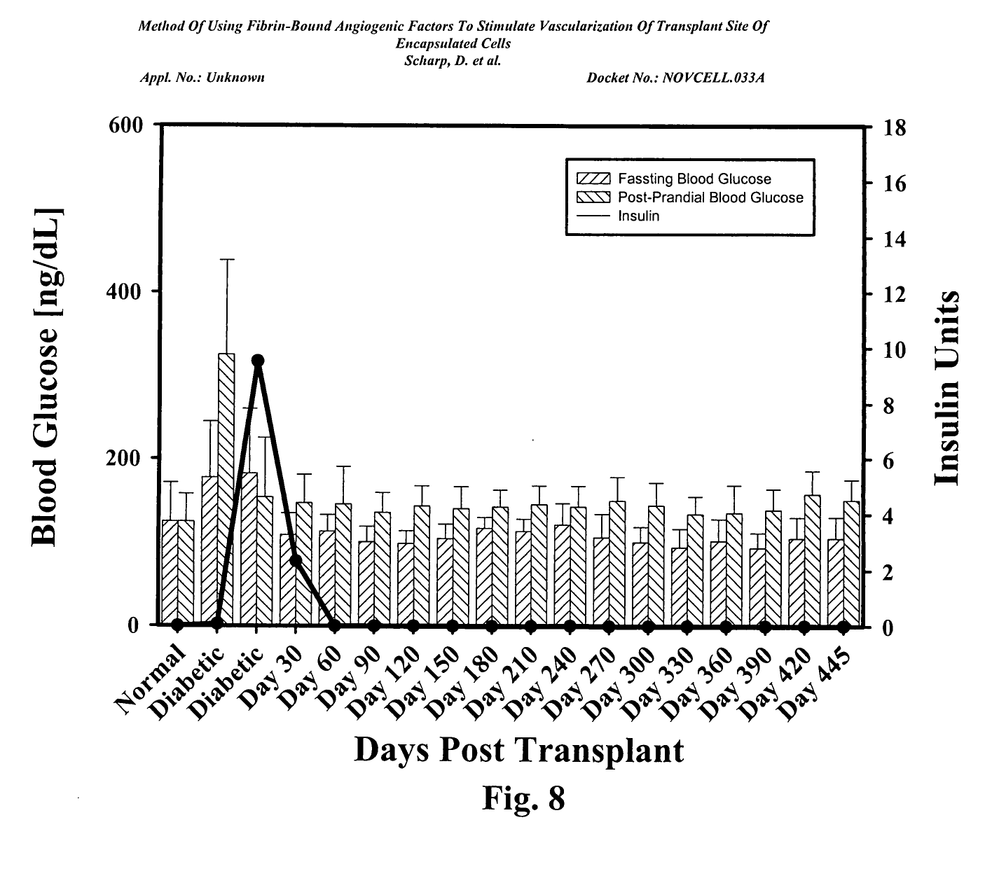

FIG. 8 illustrates the response in the recipient was rapid with a return to normoglycemia without exogenous insulin by 14 days post-implant and remained without insulin for 180 days.

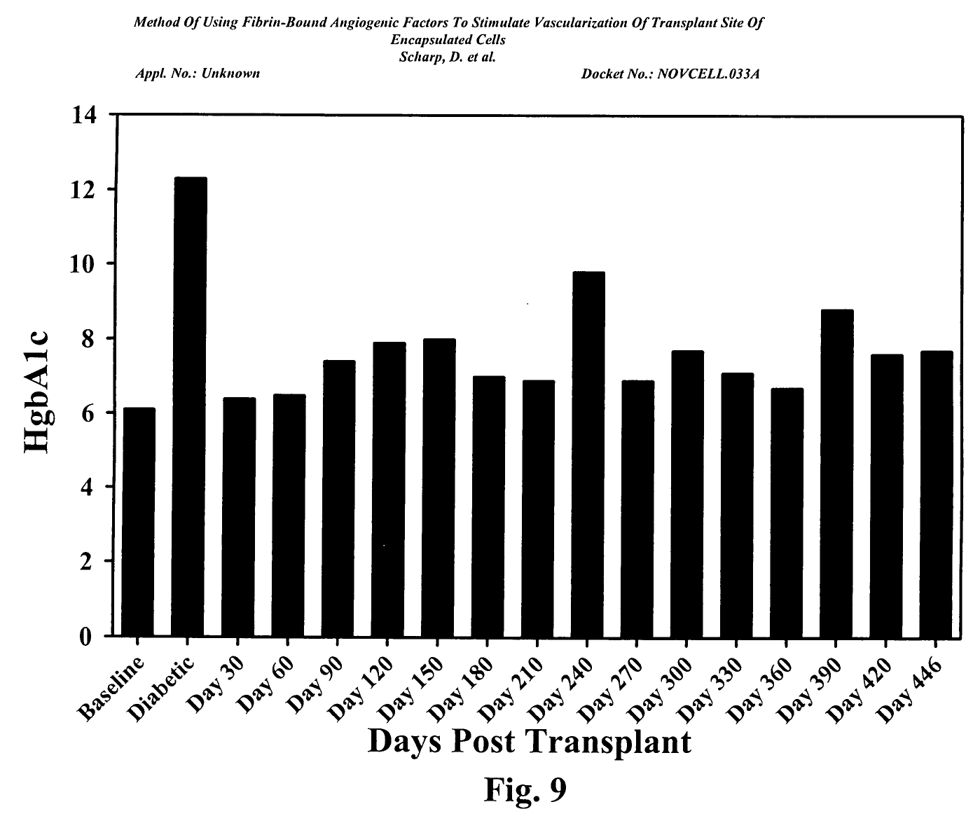

Prior to the induction of diabetes, the Hemoglobin A1c level was in the normal range at 6%. Following diabetes, it was elevated to 12%, but the Hemoglobin A1c levels returned to normal by 30 days after implant, (FIG. 9).

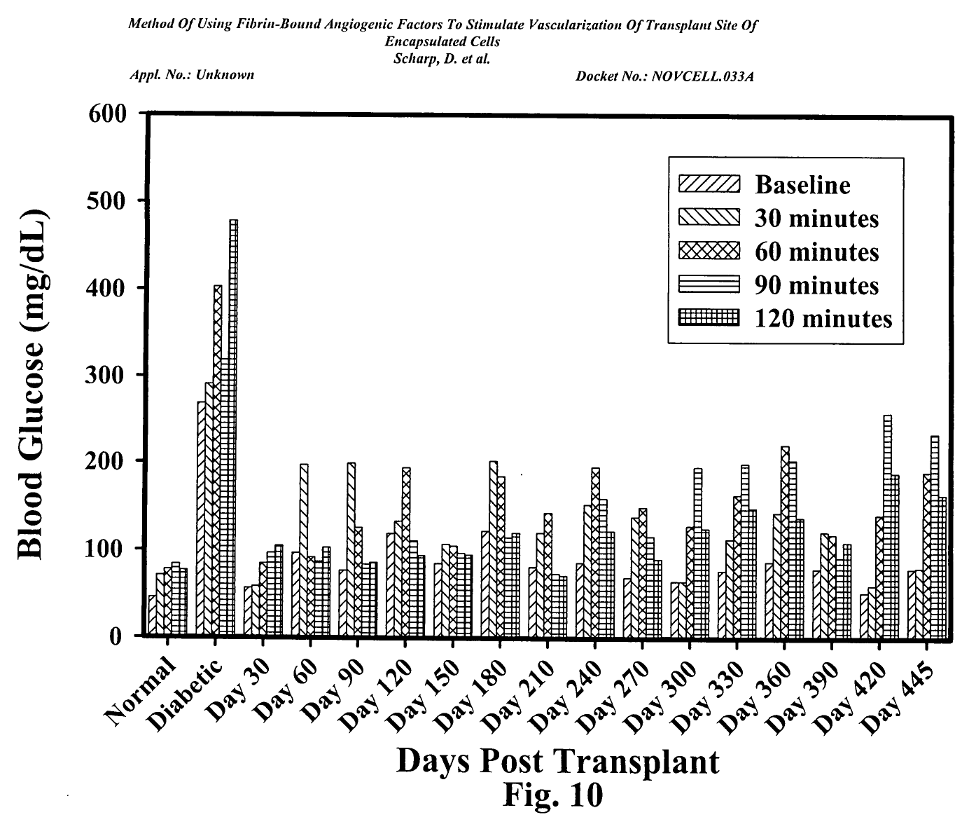

OGTT test results showed a normal response prior to the induction of diabetes and the typical diabetic response after the induction of diabetes with blood glucose values in the 400 mg/dl range (FIG. 10). Following encapsulated islet allograft implants into the enhanced subcutaneous site, the blood glucose values returned to normal at 30 days and only showed mild glucose elevation at 30 minutes during the 60 day and 90 day post-implant tests. The Day 120 test showed a delay in response with elevated glucose levels up to 60 minutes with a reduction back to normal at 120 minutes.

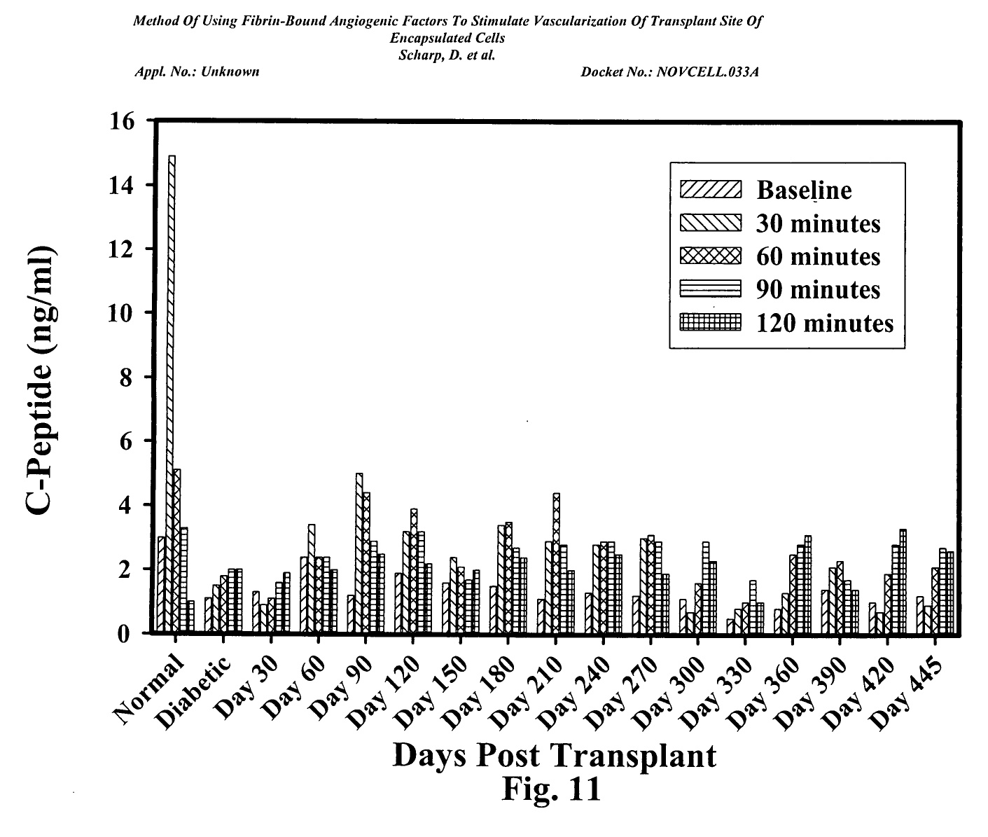

The C-peptide values were normal prior to diabetes induction and were significantly reduced after the induction of diabetes. The C-peptide values were higher than the other recipients, which suggested a milder level of diabetes but the values were elevated compared with the diabetic level (FIG. 11).

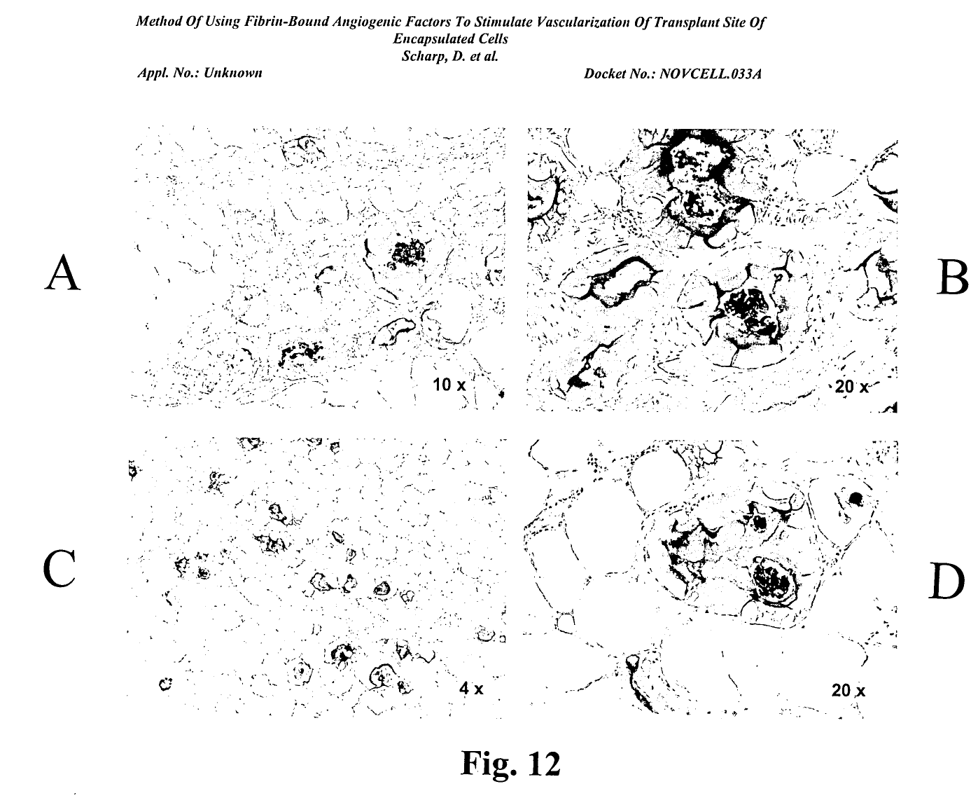

FIGS. 12A and 12B shows photomicrographs of the encapsulated islet allografts at the subcutaneous implant site at 13-14 months. The islets are viable and functional.

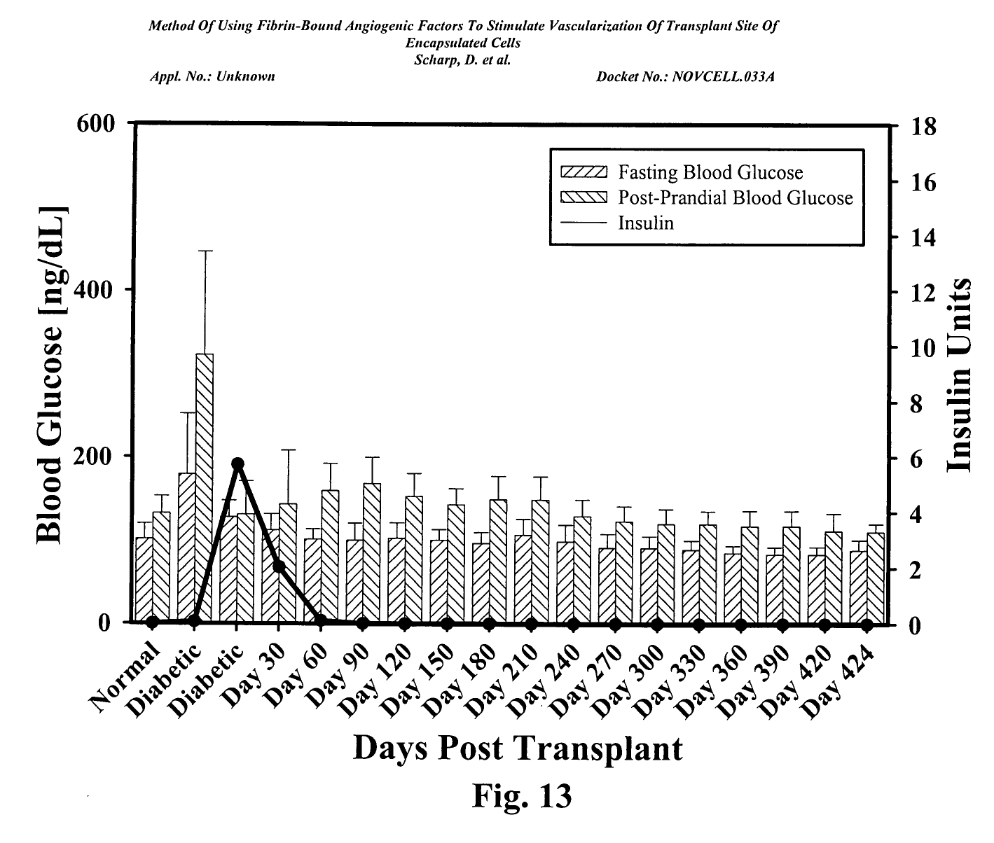

A second baboon was implanted with islets, fibrin glue and VEGF. The animal received two injections of streptozotocin to achieve diabetes, although milder than the first recipient. The animal went from 6-8 units of insulin per day to two units per day for the first 30 days (the period of cyclosporine administration). The insulin injections were stopped at the end of cyclosporine administration (FIG. 13).

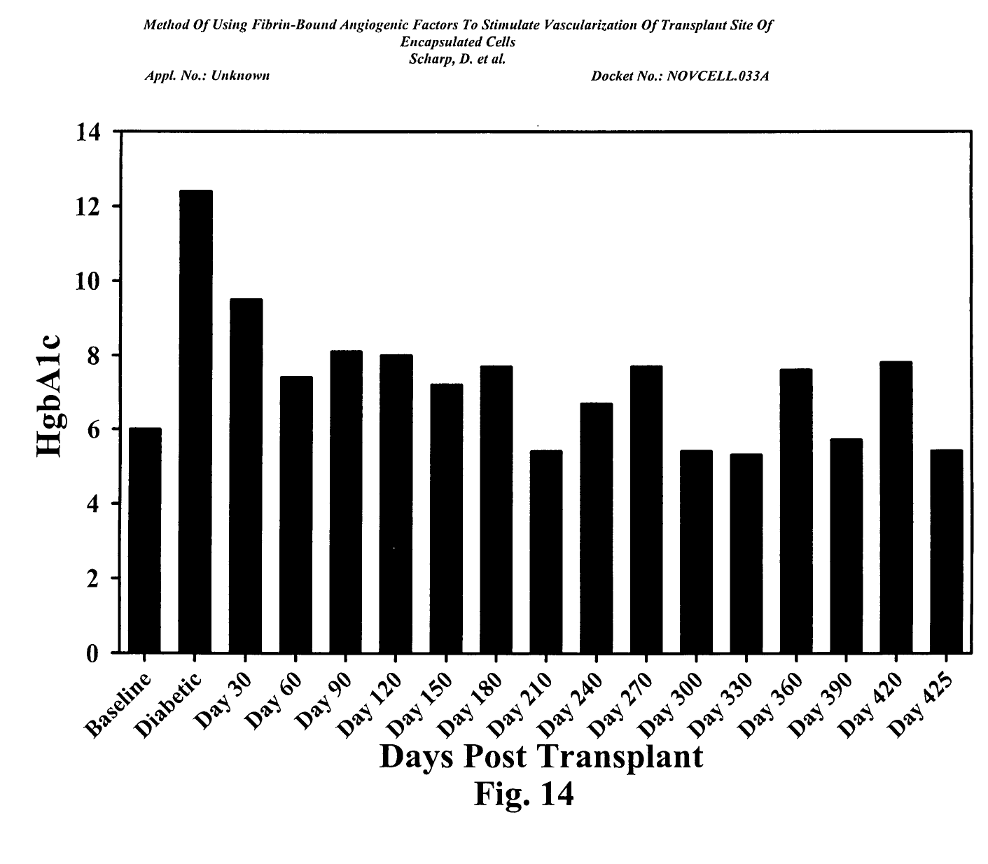

The Hemoglobin A1c levels were normal (˜6%) prior to induction of diabetes, but rose to ˜12% following diabetes induction (FIG. 14). At the end of 30 days, while this recipient was still requiring low levels of insulin, the Hemoglobin A1c level fell to ˜9%.

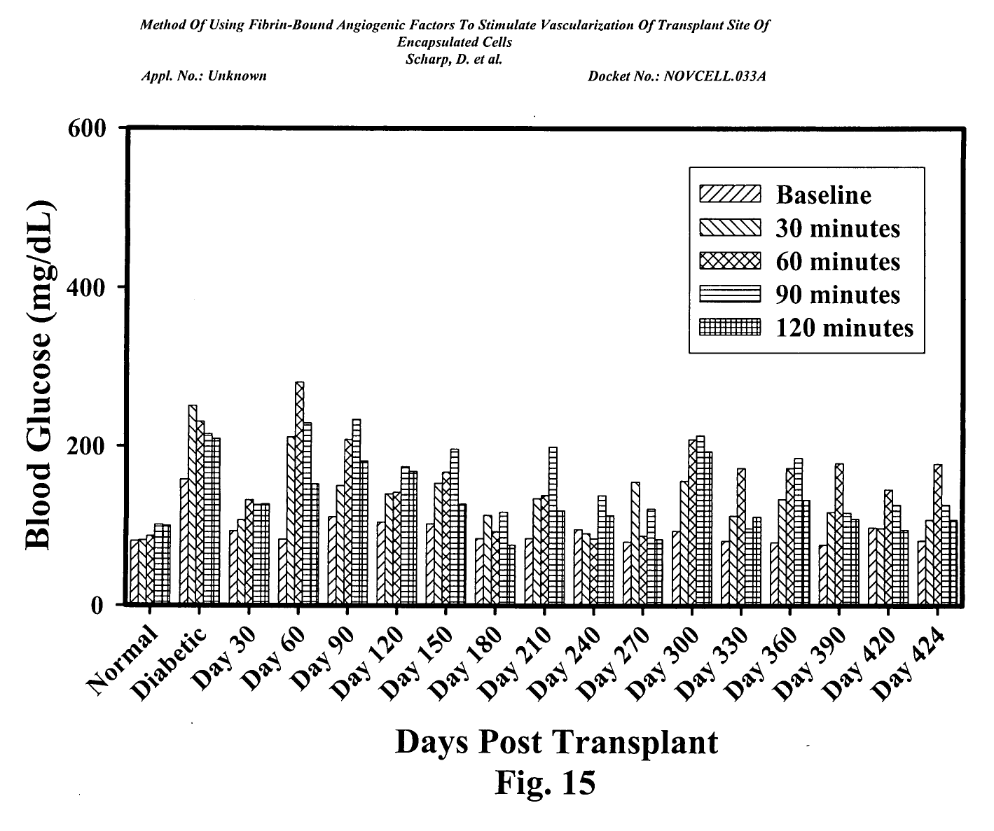

Examining the results of OGTT, a normal glucose response was achieved prior to the induction of diabetes and a hyperglycemic response while diabetic, but as expected, not as high as earlier recipients (FIG. 15). The glucose response returned to normal by 30 days after the implant even with the continuation of low dose insulin.

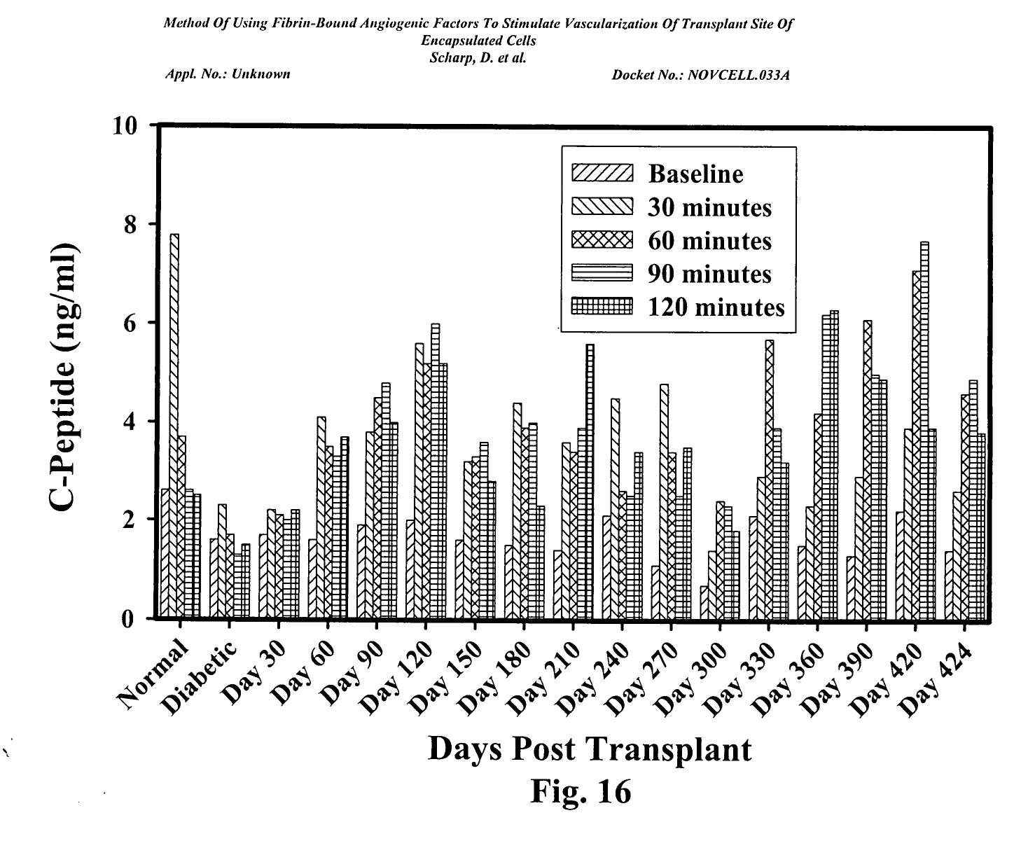

The C-peptide values during the normal period demonstrated normal C-peptide values. The diabetic period showed significantly reduced values, but more elevated than the earlier recipients (FIG. 16).

FIGS. 12C and 12D shows photomicrographs of the encapsulated islet allografts at the subcutaneous implant site at 13-14 months. The islets are viable and functional.

These findings showed the value of enhancing the subcutaneous site by the use of implanting the encapsulated islets into fibrin glue with VEGF.

### Example 4

PEG Conformally-Coated Islet Cell, TG-VEGF₁₂₁, and Fibrin Glue Preparation for Implantation and Surgical Implantation Procedure

The pancreatic islet cells were isolated and encapsulated with PEG. The encapsulated cells arrived on the morning of each implantation surgery. The islet cell, TG-VEGF₁₂₁, and Fibrin Glue compositions took up to several hours to prepare. They were prepared immediately prior to the surgical implantation procedure.

General

The islet cells were washed using standard aseptic cell culture techniques and handled in a biological safety cabinet (BSC) or laminar flow hood. The BSC or laminar flow hood was decontaminated prior to use and sterile materials and aseptic technique were used for islet cell preparation. Isopropanol alcohol was sprayed on all items (including gloved hands) prior to entering the BSC or laminar flow hood. The person handling the islet cells, TG-VEGF₁₂₁, or Fibrin Glue wore proper attire, which included disposable gown, bouffant, shoe covers, facemask, protective eyewear, sterile sleeves and sterile gloves.

Test Article Combining

Each flask was visually examined for leakage or signs of contamination. Isopropanol was sprayed on the flasks and then the flasks are placed upright in the hood. The islets were allowed to settle to the bottoms of the flasks for approximately 1 to 30 minutes. Using a sterile pipette, approximately 0.1 to 10 ml of supernatant was drawn off from each flask and combined into sterile conical tubes. The conical tubes were labeled with the implant lot number and date. The remaining supernatant was aspirated using a vacuum apparatus and sterile pipette, leaving approximately 10 to 100 ml of media with the islets. Again, the islets were allowed to settle for 1 to 60 minutes. The supernatant was aspirated leaving approximately 1 to 100 ml of media with the islets. The islets and remaining media were pooled into the appropriate number of sterile conical tubes, distributing the islets approximately evenly between the tubes. The final number of conical tubes at the end of the washing/pooling procedure was equal to the number of implant sites planned for the test article. Each flask was rinsed with Wash Solution to capture any remaining islets. This rinse medium was distributed among the conical tubes containing the islets. The conical tubes were centrifuged for approximately 15 seconds to 5 minutes at 50 to 500 g.

PEG-Conformally Coated Islet Washing

A sterile pipette was used to aspirate the supernatant from each conical tube, leaving 1 to 50 ml of media and taking care not to aspirate any of the islets. Wash Solution was added to each conical tube, cap, and gently inverted to re-suspend the islets. The conical tubes were centrifuged for 15 seconds to 5 minutes at 50 to 500 g. This was repeated for 1 to 15 washes.

If a test article was implanted, the supernatant was aspirated from the tube containing the test article, leaving 5 to 100 ml of media and taking care not to aspirate any of the islets. Excess media was removed with transfer pipettes. The test article was transferred to the surgical suite for surgical implantation.

If a test article was implanted with TG-VEGF₁₂₁/Fibrin Glue, the final aspiration removed all except approximately 50 to 900 μL of media. The islets were divided into 2 to 40 aliquots. Excess media was removed.

TG-VEGF₁₂₁ and Fibrin Glue Reagents

1. TG-VEGF₁₂₁, Receptor Ligand Technologies GmbH (RELIATech), 1 to 2 μg/μL in acetic acid buffer, stored in a freezer at −60 to −80° C.;
2. 10 to 50 mM Tris in 0.9% NaCl;
3. Fibrin Sealant, Tisseel VH, Two component Fibrin Sealant, Vapor Heated, Baxter Healthcare Corporation, product number 921030, 5.0-ml size, stored in a refrigerator at 2 to 80° C.; and
4. Tisseel VH Fibrin Sealant contained the following substances in four separate vials:
   a. Sealer Protein Concentrate (Human), Vapor Heated, freeze-dried;
   b. Fibrinolysis Inhibitor Solution (Bovine);
   c. Thrombin (Human), Vapor Heated, freeze-dried; and
   d. Calcium Chloride Solution
      TG-VEGF₁₂₁ and Fibrin Glue Preparation

The Sealer Protein Concentrate, Fibrinolysis Inhibitor Solution, Thrombin, and Calcium Chloride Solution contained in the Tisseel VH Fibrin Sealant, and the Tris buffer were warmed to 37° C. (±3° C.). The vial of Sealer Protein Concentrate was dissolved into the Fibrinolysis Solution by warming to 37° C. (±3° C.) and stirring until completely dissolved (4 to 6 ml).

The Sealer Protein Concentrate/Fibrinolysis Solution was diluted with Tris buffer using a 1:1 to 1:10 ratio of Sealer Protein Concentrate/Fibrinolysis Solution to Tris buffer to make 10 to 50 ml (±5 ml) of Sealer Protein Concentrate/Fibrinolysis Solution/Tris buffer. The solution was separated into 1 to 5 ml aliquots in cryogenic vials and maintained at 37° C. (±3° C.).

The vial of Thrombin was dissolved into the Calcium Chloride Solution by warming to 37° C. (±3° C.) and stirring until completely dissolved. 0.5 to 5 ml of the Thrombin/Calcium Chloride Solution was removed and diluted with 15 to 19.5 ml of Tris buffer. The diluted Thrombin/Calcium Chloride Solution was further diluted once more (1:5 to 1:50) with Tris buffer. The twice-diluted Thrombin/Calcium Chloride Solution was separated into aliquots in cryogenic vials and maintained at 37° C. (±3° C.). The TG-VEGF₁₂₁ was thawed and maintained at 2° to 8° C. until used.

The Sealer Protein Concentrate/Fibrinolysis Solution/Tris buffer, Thrombin/Calcium Chloride Solution, islets, and TG-VEGF₁₂₁, were transferred along with transfer pipettes, micropipettes/tips into the surgery suite.

| Treatment Group | Duration of | |
|---|---|---|
| Number | Treatment | Implanted Material |
| 1 | 3 Months | Right Side of Abdomen: |
| | | 0.9% Sodium Chloride. |
| | | Left Side of Abdomen: |
| | | TG-VEGF₁₂₁/Fibrin/Glue. |
| 2 | 3 Months | Two animals/sex: |
| | | PEG conformally-coated baboon |
| | | allogeneic islets. |
| | | Two animals/sex: |
| | | PEG conformally-coated baboon |
| | | allogeneic islets with |
| | | TG-VEGF₁₂₁/Fibrin Glue. |

Surgical Implantation with PEG Conformally-Coated Islets without TG-VEGF₁₂₁/Fibrin Glue

Each animal (a blood glucose level less than 200 mg/dL) was anesthetized and a catheter was placed into the subcutaneous site of the anterior abdomen. A Baron Suction Tube, was inserted through the implanted catheter to create a “fan shaped” area of 1 to 20 subcutaneous tracts (each approximately 1 to 6 inches in length) under the skin of the abdomen. The catheter insertion site was closed around the catheter with a purse string suture to prevent any leakage from the insertion site. PEG conformally-coated baboon allogeneic islets were drawn into a syringe, gently suspended, injected through the catheter, and deposited along the subcutaneous tracts with an even pattern of deposition throughout the tracts. One to ten milliliters was deposited into each site. The catheter was removed and the insertion site sutured closed. The dose area was marked by tattooing.

Surgical Implantation with PEG Conformally-Coated Islets with TG-VEGF₁₂₁/Fibrin Glue

Each animal (a blood glucose level less than 200 mg/dL) was anesthetized and a catheter was placed into the subcutaneous site of the anterior abdomen. A Baron Suction Tube was inserted through the implanted catheter to create a “fan shaped” area of 1 to 20 subcutaneous tracts (each approximately 1 to 6 inches in length) under the skin of the abdomen. The catheter insertion site was closed around the catheter with a purse string suture to prevent any leakage from the insertion site. Aliquots of fibrinogen solution and TG-VEGF₁₂₁ solution were combined in a ratio of 1:2 to 5:1. Aliquots of diluted thrombin and islet-containing tubes were combined in a ratio of 1:2 to 5:1. The TG-VEGF₁₂₁/fibrinogen was transferred into the islet-thrombin tube. Mixed well, injected through the catheter, and deposited along the subcutaneous tracts with an even pattern of deposition throughout approximately half of the tracts. Repeated combining TG-VEGF₁₂₁, fibrinogen, thrombin, and islets and deposited throughout the remaining tracks of the implant site. One to five TG-VEGF₁₂₁/fibrinogen/thrombin/islet aliquots were used for each implant site. One to 10 milliliters was deposited into each site. Repeated mixing of TG-VEGF₁₂₁/fibrinogen, thrombin and coated islets and deposited along the subcutaneous tracks for each of the remaining aliquots. The catheter was removed and the insertion site was sutured closed. The dose area was marked by tattooing.

Surgical Implantation with TG-VEGF₁₂₁/Fibrin Glue

Each animal (a blood glucose level less than 200 mg/dL) was anesthetized and a catheter placed into the subcutaneous site of the anterior abdomen. A Baron Suction Tube was inserted through the implanted catheter to create a “fan shaped” area of 1 to 20 subcutaneous tracts (each approximately 1 to 6 inches in length) under the skin of the abdomen. The catheter insertion site was closed around the catheter with a purse string suture to prevent any leakage from the insertion site. Aliquots of fibrinogen solution and TG-VEGF₁₂₁ solution were combined in a ratio of 1:2 to 5:1. One to five aliquots of diluted thrombin were removed and combined with the fibrinogen/TG-VEGF₁₂₁ solution. Mixed well with gentle inversions and injected along the subcutaneous tracts with an even pattern of deposition throughout the tracts. One to five TG-VEGF₁₂₁/fibrinogen/thrombin aliquots were used for each implant site. One to 10 milliliters were deposited into each site. Repeated mixing of TG-VEGF₁₂₁/fibrinogen and thrombin and deposited along the subcutaneous tracks for each of the remaining aliquots. The catheter was removed and the insertion site was sutured closed. Each site was marked by tattooing.

Surgical Implantation with Saline

Each animal (a blood glucose level less than 200 mg/dL) was anesthetized and a catheter placed into the subcutaneous site of the anterior abdomen. A Baron Suction Tube was inserted through the implanted catheter to create a “fan shaped” area of 1 to 20 subcutaneous tracts (each approximately 1 to 6 inches in length) under the skin of the abdomen. The catheter insertion site was closed around the catheter with a purse string suture to prevent any leakage from the insertion site. One to 10 milliliters of 0.9% Sodium Chloride was deposited into each site. The catheter was removed and the insertion site was sutured closed. The dose area was marked by tattooing.

### Example 5

Ischemic Muscle Implants Using Genetically Engineered Cells Producing Angiogenic Growth Factors that are Conformally Coated with PEG Coatings

Many different cell types can be genetically engineered to produce different angiogenic growth factors. These cells are human or animal fibroblasts, vascular cells, or various non-tumorigenic cell lines. The choices of angiogenic growth factors, such as VEGF, bFGF, and PDGF, are made to use as the genetically engineered cell line for encapsulation. Outcome measurements required before considering implantation into animal models with ischemic muscles are the release of the chosen angiogenic growth factor at a level presumed to provide a clinical response in the microenvironment of the ischemic muscle. If the cells were made to aggregate, then conformal coatings of these cell aggregates was done. Implantations of these encapsulated angiogenic growth factor producing cells were made in rodent models with either experimentally induced ischemic myocardium or experimentally induced ischemic limb muscles. Outcome measurements were histological demonstration of increased muscle mass and functional evidence of increased exertion of the ischemic muscle selected including cardiac muscle. Implants of these angiogenic growth factor producing cells in larger animals including humans was accomplished through vascular access and fluoroscopic control permitting direct injection in the myocardium, for example, without the need for any open surgical procedure.

It is to be understood that the foregoing descriptions are exemplary and explanatory in nature, and are intended to illustrate the invention and its preferred embodiments. Through routine experimentation, the artisan will recognize apparent modifications-and variations that may be made without departing from the spirit of the invention. Therefore, it should be clearly understood that the forms of the present invention are illustrative only and are not intended to limit the scope of the present invention.

1. A method of stimulating vascularization at a transplant site in an animal comprising:
providing a pharmaceutical composition comprising a fibrinogen, a fibrinolysis inhibitor, an angiogenic growth factor, one or more encapsulated cells, a thrombin and a divalent salt; and administering the pharmaceutical composition at said transplant site to said animal.

2. The method of claim 1, wherein the angiogenic growth factor is a modified angiogenic growth factor.

3. The method of claim 1, wherein the divalent salt is a calcium salt.

4. The method of claim 1, wherein the administration of the pharmaceutical composition is by injection.

5. The method of claim 1, wherein the angiogenic growth factor is selected from the group consisting of Angiogenin, Angiotropin, Epidermal Growth Factor (EGF), Beta Fibroblast Growth Factor (β-FGF), Fibroblast Growth Factor-2 (FGF-2), Fibroblast growth factors (FGFs), Heparin-binding EGF-like growth factor, Hepatocyte growth factor (HGF), Insulin-Like Growth Factor I (IGF-I), Interferon-gamma (IFN-gamma), Interferon-g-inducible protein-10 (IP-10), Interleukin-8 (IL-8), Macrophage inflammatory protein-1 (MIP-1), Placental growth factor (PIGF), Platelet Derived Endothelial Cell Growth Factor, Platelet factor-4 (PF-4), Platelet-derived growth factor (PDGF), platelet-derived growth factor-BB (PDGF-BB), Pleiotrophin, Transforming Growth Factor α (TGF-α), Transforming Growth Factor β (TGF-β), and Vascular Endothelial Growth Factor (VEGF).

6. The method of claim 5, wherein the angiogenic growth factor is VEGF.

7. The method of claim 1, wherein the encapsulated cell is selected from the group consisting of a macroencapsulated cell, a microencapsulated cell and a conformally coated encapsulated cell.

8. The method of claim 1, wherein the encapsulated cell is a cell derived from a stem cell.

9. The method of claim 8, wherein the derived cell is a hormone-producing cell.

10. The method of claim 9, wherein the hormone-producing cell is an insulin-producing cell.

11. The method of claim 1, wherein the encapsulated cell is from the Class Mammalia.

12. The method of claim 11, wherein the encapsulated cell is human.

13. The method of claim 1, further comprising administering, individually or in combination, an immunosuppressant and an anti-inflammatory agent.

14. The method of claim 13, further comprising administering, individually or in combination, the immunosuppressant and anti-inflammatory agent for a period of no more than 6 months from the time of treatment.

15. The method of claim 14, further comprising administering, individually or in combination, the immunosuppressant and anti-inflammatory agent for a period of no more than 1 month from the time of treatment.

16. The method of claim 1, wherein the animal is from the Class Mammalia.

17. The method of claim 16, wherein the animal is Human.

18. The method of claim 1, wherein the pharmaceutical composition additionally comprises Factor XIII.

19. The method of claim 18, wherein a substrate for a transglutaminase activity of Factor XIII is attached to the angiogenic growth factor.

20. A method of preparing a pharmaceutical composition comprising:
(a) preparing a solution comprising a fibrinogen, a fibrinolysis inhibitor, and an angiogenic growth factor to form a fibrinogen/fibrinolysis inhibitor/angiogenic growth factor solution;
(b) adding at least one encapsulated cell to the fibrinogen/fibrinolysis inhibitor/angiogenic growth factor solution; and
(c) adding a thrombin into the encapsulated cell/fibrinogen/fibrinolysis inhibitor/angiogenic growth factor solution of step (b) to produce a pharmaceutical composition comprising a thrombin, at least one encapsulated cell, fibrinogen, fibrinolysis inhibitor, and angiogenic growth factor.

21. A method of stimulating vascularization at a transplant site in an animal comprising administering the pharmaceutical composition prepared by the method of claim 20 to said animal.

22. The method of claim 20, wherein the solution of step (a) additionally comprises Factor XIII.

23. The method of claim 20, wherein the thrombin added in step (c) is in a solution additionally comprising a divalent salt.

24. The method of claim 23, wherein the divalent salt is a calcium salt.

25. A method of preparing a pharmaceutical composition comprising:
(a) preparing a solution comprising a thrombin and an angiogenic growth factor to form a thrombin/angiogenic growth factor solution;
(b) adding at least one encapsulated cell to the thrombin/angiogenic growth factor solution; and
(c) adding a fibrinogen and a fibrinolysis inhibitor into the encapsulated cell/thrombin/angiogenic growth factor solution of step (b) to produce a pharmaceutical composition comprising a fibrinogen, a fibrinolysis inhibitor, at least one encapsulated cell, thrombin and an angiogenic growth factor.

26. A method of stimulating vascularization at a transplant site in an animal comprising administering the pharmaceutical composition prepared by the method of claim 25 to said animal.

27. The method of claim 25, wherein the thrombin added in step (a) is in a solution additionally comprising a divalent salt.

28. The method of claim 27, wherein the divalent salt is a calcium salt.

29. The method of claim 25, wherein the solution of step (c) additionally comprises Factor XIII.

30. A method of stimulating vascularization at a transplant site in an animal comprising:
placing a first solution comprising fibrinogen, fibrinolysis inhibitor, angiogenic growth factor and at least one encapsulated cell into a first barrel of a syringe; and
placing a second solution comprising thrombin into a second barrel of a syringe;
and injecting the first and second solutions into an injection site on said animal.

31. The method of claim 30, wherein the injection site on the animal is subcutaneous, intraperitoneal, intramuscular, intra-omental, or into an organ.

32. The method of claim 31, wherein the injection site on the animal is a subcutaneous site.

33. A method of stimulating vascularization at a transplant site in an animal comprising:
placing a first solution comprising thrombin, angiogenic growth factor and at least one encapsulated cell into a first barrel of a syringe;
placing a second solution comprising fibrinogen and fibrinolysis inhibitor into a second barrel of a syringe; and
injecting the first and second solutions into an injection site on said animal.

34. The method of claim 33, wherein the injection site on the animal is subcutaneous, intraperitoneal, intramuscular, intra-omental, or into an organ.

35. The method of claim 34, wherein the injection site on the animal is a subcutaneous site.

36. A pharmaceutical composition comprising: a fibrinogen, a fibrinolysis inhibitor, an angiogenic growth factor, one or more encapsulated cells, a thrombin and a divalent salt.

37. The pharmaceutical composition of claim 36, wherein the angiogenic growth factor is selected from the group consisting of Angiogenin, Angiotropin, Epidermal Growth Factor (EGF), Beta Fibroblast Growth Factor (β-FGF), Fibroblast Growth Factor-2 (FGF-2), Fibroblast growth factors (FGFs), Heparin-binding EGF-like growth factor, Hepatocyte growth factor (HGF), Insulin-Like Growth Factor I (IGF-I), Interferon-gamma (IFN-gamma), Interferon-g-inducible protein-10 (IP-10), Interleukin-8 (IL-8), Macrophage inflammatory protein-1 (MIP-1), Placental growth factor (PIGF), Platelet Derived Endothelial Cell Growth Factor, Platelet factor-4 (PF-4), Platelet-derived growth factor (PDGF), platelet-derived growth factor-BB (PDGF-BB), Pleiotrophin, Transforming Growth Factor α (TGF-α), Transforming Growth Factor β (TGF-β), and Vascular Endothelial Growth Factor (VEGF).

38. The pharmaceutical composition of claim 37, wherein the angiogenic growth factor is a modified angiogenic growth factor.

39. The pharmaceutical composition of claim 38, wherein the modified angiogenic growth factor is α-2PI₁₋₈-VEGF₁₂₁.

40. The pharmaceutical composition of claim 36, wherein the encapsulated cell is selected from the group consisting of a macroencapsulated cell, a microencapsulated cell and a conformally coated encapsulated cell.

41. The method of claim 36, wherein the divalent salt is a calcium salt.

42. The pharmaceutical composition of claim 36, wherein the encapsulated cell is a cell derived from a stem cell.

43. The pharmaceutical composition of claim 42, wherein the derived cell is a hormone-producing cell.

44. The pharmaceutical composition of claim 43, wherein the hormone-producing cell is an insulin-producing cell.

45. The pharmaceutical composition of claim 36, wherein the encapsulated cell is from the Class Mammalia.

46. The pharmaceutical composition of claim 45, wherein the encapsulated cell is human.
